(19) United States
(12) Patent Application Publication
(10) Pub. No.: US 2020/0129545 A1
(43) Pub. Date: Apr. 30, 2020

(54) METHODS OF TREATMENT WITH MIXED METAL COMPOUND

(71) Applicant: OPKO IRELAND GLOBAL HOLDINGS, LTD., Grand Cayman (KY)

(72) Inventor: P. Martin Petkovich, Kingston (CA)

(21) Appl. No.: 16/664,698

(22) Filed: Oct. 25, 2019

Related U.S. Application Data
(60) Provisional application No. 62/750,791, filed on Oct. 25, 2018.

(51) Int. Cl. A61K 33/26; A61P 3/14; A61P 9/14

(52) U.S. Cl. CPC A61K 33/26; A61P 9/14; A61P 3/14

(57) ABSTRACT

A method treating and/or preventing vascular calcification can include administering a mixed metal compound to a subject in need thereof.

### CROSS-REFERENCE TO RELATED APPLICATION

The benefit of priority of U.S. Provisional Patent Application No. 62/750,791 filed Oct. 25, 2018, is hereby claimed and the disclosure is incorporated herein by reference in its entirety.

### BACKGROUND

### Field of the Disclosure

The disclosure relates generally to methods of using mixed metal compounds, uses of mixed metal compounds, and mixed metal compounds for particular uses, including pharmaceutical uses, e.g. in preventing or reducing vascular calcification and in lowering serum and/or plasma parathyroid hormone (PTH) levels.

### Brief Description of Related Technology

Vascular calcification (VC) is the pathological deposition of mineral in the vascular system. It has a variety of forms, which include intimal calcification and medial calcification, as well as presence in the valves of the heart. Traditional risk factors for vascular calcification include age, male gender, smoking, diabetes, hypertension dyslipidemia and other atherosclerotic risk factors. Patients with vascular calcification are at higher risk for adverse cardiovascular events.

Hyperphosphatemia is commonly found in patients with chronic kidney disease. Cardiovascular disease is the most common cause of death in patients with chronic kidney disease and vascular calcification can be a strong predictor of cardiovascular risk. In CKD patients, disordered mineral metabolism may initiate and/or promote progression of vascular calcification. Important factors regulating mineral metabolism are calcium, phosphate, parathyroid hormone (PTH), vitamin D, and fibroblast group factor-23 (FGF23).

Vascular calcification can also be found in patients with recurrent urolithiasis, such as subjects with idiopathic hypercalciuria. (Ha, 51 Korean J. Urol 54-49 (201).

### SUMMARY

One aspect of the disclosure is a method of preventing vascular calcification comprising administering to a subject in need thereof an effective amount of a mixed metal compound described herein. The subject in need thereof can be a subject having hyperphosphatemia. The subject in need thereof can be a subject having elevated phosphate levels. The subject in need thereof can be a subject having chronic kidney disease (CKD). The subject in need thereof can be a subject having elevated FGF23. The subject in need thereof can be a subject having hyperphosphaturia. The subject can have hyperparathyroidism. The hyperparathyroidism can be secondary to the chronic kidney disease. The subject in need thereof can have any combination of the foregoing conditions.

The subject in need thereof can be a non-CKD subject having elevated FGF23 and/or hyperphosphaturia. The subject in need thereof can be a non-CKD subject having urolithiasis. The subject in need thereof can be a non-CKD subject having idiopathic hypercalciuria. The subject in need thereof can be a non-CKD subject having hyperphosphatemia. The subject in need thereof can have any combination of the foregoing conditions.

In any of the methods disclosed herein the subject can be receiving hemodialysis therapy.

Another aspect of the disclosure is a method of lowering serum or plasma parathyroid hormone level comprising administering to a subject in need therein an effective amount of a mixed metal compound described herein.

Another aspect of the disclosure is a method of preventing an increase in serum or plasma parathyroid hormone level comprising administering to a subject in need therein an effective amount of a mixed metal compound described herein.

Another aspect of the disclosure is a method of both preventing vascular calcification and lowering serum or plasma parathyroid hormone level comprising administering to a subject in need therein an effective amount of a mixed metal compound described herein.

Another aspect of the disclosure is a method of both preventing vascular calcification and preventing an increase in serum and/or plasma parathyroid hormone level comprising administering to a subject in need therein an effective amount of a mixed metal compound described herein.

Another aspect of the disclosure is use of a mixed metal compound described herein for any treatment or method described herein, or for manufacture of a medicament for a treatment or use described herein.

Another aspect of the disclosure is a composition comprising a mixed metal compound for a use, treatment, or method described herein, or for manufacture of a medicament for a use, treatment, or method described herein. For example, the composition can include a mixed metal compound described herein and an excipient, e.g. in tablet or liquid form as described herein.

In any aspect of a method, use, or article described herein, one or more additional features can be selected from the various embodiments described herein, including in the Example provided below. For example, a subject can be a human patient. The subject in need of therapy can have Chronic Kidney Disease. The subject in need of therapy can have Chronic Kidney Disease Stage 3-5. The subject in need of therapy can have Chronic Kidney Disease Stage 3-4. The subject in need of therapy can have Chronic Kidney Disease Stage 5 (a.k.a. End Stage Renal Disease). The subject in need of therapy can have Chronic Kidney Disease and be receiving hemodialysis therapy. The subject in need of therapy can have hyperparathyroidism. The subject in need of therapy can have hyperparathyroidism secondary to Chronic Kidney Disease. The subject in need of therapy can have hyperphosphatemia. The subject in need of therapy can have hyperparathyroidism and hyperphosphatemia. The method can include both decreasing serum phosphate and increasing serum magnesium concentrations. The method can include decreasing serum phosphate to an extent that the subject no longer has hyperphosphatemia. The method can include not significantly affecting serum creatinine concentration. The method can include not significantly affecting serum calcium concentration. The method can include reducing serum and/or plasma parathyroid hormone concentration by 16% or more. The method can include reducing serum and/or plasma parathyroid hormone concentration by 30% or more, or at least 31%. The method can include preventing calcification in arterial tissue. The method can include preventing calcification in heart tissue. The method can include preventing calcification in one or more tissues, including arteries and heart tissues including but not limited to aortic arch, carotid, mesenteric (incl. superior), aorta (incl. thoracic and ascending), iliac (including 1. iliac), femoral (including r.fem and l.fem), celiac, pudendal (incl. l.pudendal), and renal (including r.renal and l.renal). The method can include preventing calcification in one or more tissues, including arteries and heart tissues including but not limited to the aorta, carotid, distal, and pudendal. The method can include reducing the degree of vascular calcification, compared to untreated subjects, by at least 30%, or at least 44%, or at least 52%, or at least 66%.

For the compositions and methods described herein, optional features, including but not limited to components, compositional ranges thereof, substituents, conditions, and steps, are contemplated to be selected from the various aspects, embodiments, and examples provided herein.

Further aspects and advantages will be apparent to those of ordinary skill in the art from a review of the following detailed description, taken in conjunction with the drawings. While the methods, uses, and articles are susceptible of embodiments in various forms, the description hereafter includes specific embodiments with the understanding that the disclosure is illustrative, and is not intended to limit the invention to the specific embodiments described herein.

### BRIEF DESCRIPTION OF THE DRAWINGS

FIG. 1 is a schematic illustration of a comparative study of methods of the disclosure to a control method.

FIGS. 2A and 2B are graphs showing serum creatinine as a function time (weeks on study);

FIGS. 2C and 2D are graphs showing serum phosphate as a function of time (weeks on study);

FIGS. 2E and 2F are graphs showing serum calcium as a function of time (weeks on study);

FIGS. 3A and 3B are graphs showing serum phosphate as a function time (days);

FIGS. 3C and 3D are graphs showing serum magnesium as a function of time (days);

FIGS. 3E and 3F are graphs showing serum calcium as a function of time (days).

FIGS. 4A and 4B are graphs showing parathyroid hormone levels as a function of time (days);

FIGS. 5A and 5B are graphs showing FGF23 levels as a function of time (days);

FIGS. 6A to 6F are graphs showing serum vitamin D metabolite levels in the comparative study;

FIGS. 7A and 7B are graphs showing tissue phosphate levels in the comparative study;

FIGS. 7C and 7D are graphs showing tissue calcium levels in the comparative studies;

FIGS. 7E and 7F are graphs showing percent calcification in the comparative study.

FIG. 8 is a graph showing average ratio of magnesium to phosphate to average phosphate, with the inset showing the data used for determining the average values;

FIG. 9A is a graph showing average calcium as a function of average phosphate;

FIG. 9B is a graph showing average magnesium as a function of average phosphate; and

FIGS. 10A and 10B are graphs showing the ratio of tissue magnesium:phosphate in various regions of the tested subjects of the comparative study.

### DETAILED DESCRIPTION

Hyperphosphatemia, common in chronic kidney disease (CKD), is linked to vascular calcification (VC), which further increases cardiovascular risk. Phosphate shows preferential deposition in the vasculature in CKD. Serum phosphate concentrations are dependent on phosphate absorption from diet (positive correlation) and severity of CKD (positive correlation). PTH is elevated with severe CKD and increased phosphate. Vascular calcification is dependent on the severity of CKD and phosphate absorption from diet.

Both serum phosphorus and magnesium levels correlate with cardiovascular mortality. In vitro and in vivo studies have suggested a protective role of magnesium against vascular calcification through multiple molecular mechanisms. Observational studies in hemodialysis patients have suggested that the protective effect of increasing serum magnesium is additive to that of lowering serum phosphorus.

Mixed metal compounds, related compositions including mixed metal compounds (e.g. tablet and liquid formulations), methods of making such compounds and compositions, and related uses are described in U.S. Pat. Nos. 6,926,912, 7,799,251, 8,568,792, 9,242,869, 9,168,270, 9,907,816, 9,314,481, 9,066,917, and 9,566,302, and U.S. Patent Application Publication Nos. 2008/0206358, 2010/0125770, and 2010/0203152, and the disclosures thereof are incorporated by reference herein. Such compounds have been shown to have phosphate binding ability.

Without intending to be bound by theory, it is further believed that mixed metal compounds containing magnesium as a bivalent metal can release a portion of the bivalent metal during phosphate binding. It has been surprisingly found that absorption of magnesium from administration of mixed metal compounds disclosed herein resulted in preferential absorption of magnesium by vascular tissues. It is believed that such preferential absorption by the vascular tissue, plus the resulting increase in accumulation of magnesium relative to phosphate inhibited or reduced vascular calcification through both the protective function of magnesium and reduction of phosphate. One such mixed metal compound is an iron magnesium hydroxy carbonate with the general formula [Mg₄Fe₂(OH)₁₂].CO₃.4H₂O, commonly referred to as fermagate. Fermagate is a calcium-free, magnesium-releasing phosphate binder that controls hyperphosphatemia.

A method of treating vascular calcification can include administering any one or more of the mixed metal compounds as described herein to a subject in need thereof, wherein the subject has chronic kidney disorder. The method can include administering a mixed metal compound comprising at least magnesium as the bivalent metal. The method can include administering a mixed metal compound in which the bivalent metal is magnesium.

A method of treating vascular calcification can include administering any one or more of the mixed metal compounds as described herein to a subject in need thereof, wherein the subject has hyperphosphatemia. The subject can further have chronic kidney disorder. The subject can alternatively be a non-chronic kidney disorder subject. The method can include administering a mixed metal compound comprising at least magnesium as the bivalent metal. The method can include administering a mixed metal compound in which the bivalent metal is magnesium.

A method of treating vascular calcification can include administering any one or more of the mixed metal compounds as described herein to a subject in need thereof, wherein the subject has elevated FGF23 and/or hyperphosphaturia. The subject can further have chronic kidney disorder. The subject can alternatively be a non-chronic kidney disorder subject. The method can include administering a mixed metal compound comprising at least magnesium as the bivalent metal. The method can include administering a mixed metal compound in which the bivalent metal is magnesium.

A method of treating vascular calcification can include administering any one or more of the mixed metal compounds as described herein to a subject in need thereof, wherein the subject has urolithiasis. The subject can have recurrent urolithiasis. The subject can further have idiopathic hypercalciuria. The subject can alternatively be a non-chronic kidney disorder subject. The method can include administering a mixed metal compound comprising at least magnesium as the bivalent metal. The method can include administering a mixed metal compound in which the bivalent metal is magnesium.

Magnesium plays an important role in mineral metabolism. It is believed that decreased serum magnesium levels are associated with vascular calcification in End Stage Renal Disease.

Thus, one aspect of the disclosure is a method of preventing vascular calcification comprising administering to a subject in need therein an effective amount of a mixed metal compound described herein, optionally fermagate.

Another aspect of the disclosure is a method of lowering serum and/or plasma parathyroid hormone level comprising administering to a subject in need therein an effective amount of a mixed metal compound described herein, optionally fermagate.

Another aspect of the disclosure is a method of preventing an increase in serum and/or plasma parathyroid hormone level comprising administering to a subject in need therein an effective amount of a mixed metal compound described herein, optionally fermagate.

Another aspect of the disclosure is a method of both preventing vascular calcification and lowering serum and/or plasma parathyroid hormone level comprising administering to a subject in need therein an effective amount of a mixed metal compound described herein, optionally fermagate.

For example, parathyroid hormone (PTH) can be reduced by at least about 16%, about 30% or about 31%.

Another aspect of the disclosure is a method of both preventing vascular calcification and preventing an increase in serum and/or plasma parathyroid hormone level comprising administering to a subject in need therein an effective amount of a mixed metal compound described herein, optionally fermagate.

In any of the methods disclosed herein, serum calcium concentration can remain substantially unchanged or unaffected by the administration of the mixed metal compound.

In any of the methods disclosed herein, serum creatinine concentration can be substantially unchanged or unaffected by the administration of the mixed metal compound.

In any of the methods disclosed herein, serum phosphate can be reduced. In various embodiments in which the mixed metal compound contains magnesium, serum magnesium can be increased and serum phosphate can be reduced. In such embodiments a ratio of magnesium:phosphate accumulation in vascular tissue can increase.

In any of the methods disclosed herein, calcification can be treated, reduced, and/or prevented in any one or more of heart tissue or arterial tissue. For example, vascular calcification can be treated, reduced, and/or prevented in any one or more of the aorta, caratoids, distal arteries, coronary CMR, and pudendals.

The methods, uses, and articles are contemplated to include embodiments including any combination of one or more of the additional optional elements, features, and steps further described below (including those shown in the figures and described in the Example), unless stated otherwise.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

As used herein, the term “comprising” indicates the potential inclusion of other agents, elements, steps, or features, in addition to those specified.

A subject treated herein or the subject of a use described herein can be a vertebrate, or a mammal, and can be a human patient.

The subject in need of therapy can have Chronic Kidney Disease. The subject in need of therapy can have Chronic Kidney Disease Stage 3-5. The subject in need of therapy can have Chronic Kidney Disease Stage 3-4. The subject in need of therapy can have Chronic Kidney Disease Stage 5 or End Stage Renal Disease. The subject in need of therapy can have Chronic Kidney Disease and receiving hemodialysis therapy. The subject in need of therapy can have hyperparathyroidism secondary to Chronic Kidney Disease. The subject in need of therapy can have hyperphosphatemia. The subject in need of therapy can have hyperphosphatemia, alone or in addition to Chronic Kidney Disease and/or hyperparathyroidism. The subject in need of therapy can have hyperphosphatemia and hyperparathyroidism, optionally secondary hyperparathyroidism.

The method can include both decreasing serum phosphate and increasing serum magnesium concentrations. The method can include decreasing serum phosphate to an extent that the subject no longer has hyperphosphatemia. The method can include not significantly affecting serum creatinine concentration. The method can include not significantly affecting serum calcium concentration.

The method can include reducing serum and/or plasma parathyroid hormone concentration by 16% or more. The method can include reducing serum and/or plasma parathyroid hormone concentration by 30% or more, or at least 31%.

The method can include preventing calcification in arterial tissue. The method can include preventing calcification in heart tissue. The method can include preventing calcification in one or more tissues, including arteries and heart tissues including but not limited to aortic arch, carotid, mesenteric (incl. superior), aorta (incl. thoracic and ascending), iliac (including 1. iliac), femoral (including r.fem and l.fem), celiac, pudendal (incl. l.pudendal), and renal (including r.renal and l.renal). The method can include preventing calcification in one or more tissues, including arteries and heart tissues including but not limited to the aorta, carotid, coronary CMR, distal, and pudendal. The method can include reducing the degree of vascular calcification, compared to untreated subjects, by at least 30%, or at least 44%, or at least 52%, or at least 66%.

Methods of the disclosure can include administration of the mixed metal compound can be adjusted to achieve a target serum phosphorus concentration of 2.5 to 4.5 mg/dL (0.8 to 1.45 mmol/L). For example, a mixed metal compound dosage can be titrated by 500 mg tid every two weeks for up to 10 weeks to achieve the desired target serum phosphate concentration and up to a maximum dose of 3000 mg tid. For example, an initial mixed metal compound dose of 500 mg may be given to patients with a serum phosphorus concentration of ≥5.5-7.5 mg/dL (≥1.78-2.42 mmol/L), and an initial mixed metal compound dose of 1000 mg may be given to patients with a serum phosphorous concentration of >7.5 mg/dL (>2.42 mmol/L)

After the target serum phosphorus concentration is reached or the subject has completed week 10 of titration and serum phosphorus has decreased by a minimum of 1.0 mg/dL (0.32 mmol/L), the dose may be (i) increased monthly in increments of 500 mg tid up to a maximum of 3000 mg if serum phosphorus is >4.5 mg/dL; (ii) decreased monthly in increments of 500 mg if serum phosphorus is <2.5 mg/dL, or (iii) maintained to achieve a serum phosphorus level of 2.5 to 4.5 mg/dL (0.8 to 1.45 mmol/L).

In embodiments of methods disclosed herein, the mixed metal compound can be administered in amounts in a range of 0.1 to 500, or from 1 to 200, mg/kg body weight of mixed metal compound as active compound (alone, or in any formulation type) are contemplated for administration daily to obtain the desired results. Nevertheless, it may be necessary from time to time to depart from the amounts mentioned above, depending on the body weight of the patient, the method of application, the animal species of the patient and its individual reaction to the drug or the kind of formulation or the time or interval in which the drug is applied. In special cases, it may be sufficient to use less than the minimum amount given above, while in other cases the maximum dose may have to be exceeded. For a larger dose, it may be advisable to divide the dose into several smaller single doses. Ultimately, the dose will depend upon the discretion of the attendant physician. Administration soon before meals, e.g. within one hour before a meal or taken with food is contemplated for one type of embodiment.

A single solid unit dose for human adult administration can comprise from 1 mg to 1 g, or from 10 mg to 800 mg of mixed metal compound, for example.

In any of the methods of the disclosure herein, the mixed metal compound can be administered alone or in combination with one or more additional active agents. The one or more additional active agents can be for example, active agents for treating any one of more of the conditions identified herein which a subject may have and which may be associated with or lead to vascular calcification, or which are treating other underlying conditions in the patient.

For example, for subjects with chronic kidney disease, the mixed metal compound may be administered in combination with Vitamin D therapy. The Vitamin D therapy can be, for example, one or more of Rayaldee, 25(OH)D₃, or other vitamin D natural compounds or synthetic analogs. Any vitamin D compound suitable for prophylactic and/or therapeutic use, and combinations thereof, are contemplated for use in the methods of the disclosure in combination with the phosphate binding mixed metal compounds. Vitamin D prehormones, prohormones, active vitamin D hormones, and other metabolites and synthetic analogs of Vitamin D are also useful as active compounds and can be used in combination therapies in the methods of the disclosure. Specific examples include, but are not limited to, Vitamin D₃ (cholecalciferol), Vitamin D₂ (ergocalciferol), 25-hydroxyvitamin D₃, 25-hydroxyvitamin D₂, 1α,25-dihydroxyvitamin D₃ (Calcitriol), 1α,25-dihydroxyvitamin D₂, 1α,25-dihydroxyvitamin D₄, and vitamin D analogs (including all hydroxy and dihydroxy forms), including 1,25-dihydroxy-19-nor-vitamin D₂ (Paricalcitol) and 1α-hydroxyvitamin D₃ (Doxercalciferol).

Chronic kidney disease subjects may also be administered, in addition to the phosphate binding mixed metal compounds, one or more of blood pressure medications, cholesterol medications, erythropoietin, diuretics, calcium supplements, Vitamin D therapy, and vitamin D to treat conditions and symptoms associated with the chronic kidney disease. For example, a method can include administration, in addition to the phosphate binding mixed metal compounds, of one or more of a vitamin D therapy, such as described above, calcimimetics, calcium salts, nicotinic acid, iron, calcium salts, glycemic and hypertension control agents, antineoplastic agents, inhibitors of CYP24, and inhibitors other cytochrome P450 enzymes that can degrade vitamin D agents. Such actives may be administered in combination with the mixed metal compound in various embodiments.

### Measuring and Monitoring Vascular Calcification

In any of the embodiments herein, vascular calcification can be measured and/or monitored using any known methods. Computed tomography (CT) of the aorta or coronary arteries is commonly used. Radiography of the lateral abdomen (abdomen aorta) or chest (aortic arch) and the hand can be used to detect the presence or absence of vascular calcification. Echocardiogram (ECG) can also be used to detect calcification, for example, in the mitral annulus, aortic valve leaflets, and aortic root. Low does, non-ECG-synchronized and non-contrast-enhanced CT scans of the chest and abdomen using either multi-detector row scanners or electron-beam scanners can also be used to assess cardiovascular calcification.

### Mixed Metal Compounds

The mixed metal compounds and related compositions for use herein will now be described in additional detail. As noted above, mixed metal compounds or formulations thereof, as described in U.S. Pat. Nos. 6,926,912, 7,799,251, 8,568,792, 9,242,869, 9,168,270, 9,907,816, 9,314,481, 9,066,917, and 9,566,302, and U.S. Patent Application Publication Nos. 2008/0206358, 2010/0125770, and 2010/0203152 can be used in the methods of the disclosure.

Mixed metal compounds provide unique challenges in using inorganic material for pharmaceutical use. For example, use of mixed metal compound for attaining therapeutic effects (or other pharma functional use) may depend, for example, on surface processes such as physisorption (ion-exchange) and chemisorption (formation of a chemical bond) which is atypical for a drug; the therapeutic activity of most drugs are based on organic compounds which are typically more soluble.

Yet further, high daily and repeated long-term (chronic) dosages are required for kidney patients but their total daily pill count requires a low tablet burden due to restricted fluid intake. Consequently, high dosage of drug substance is required in final product (e.g. tablet) and the final product is therefore very sensitive to the properties of the mixed metal compound drug substance, unlike normal formulations. This means that the properties of the tablet, including key physical properties, and the tablet manufacturing processes, such as granulation, are often primarily influenced by the properties of the mixed metal compound active substance rather than solely by those of the excipients. In order to be able to manufacture a pharmaceutical product comprising such significant quantities of mixed metal compound with the control and consistency necessary for pharmaceutical use, a means of controlling an array of opposing chemical and physical properties of the mixed metal compound is need such as disclosed in WO 2011/015859.

Mixed metal compounds exist as so-called “Layered Double Hydroxide” (LDH) which is used to designate synthetic or natural lamellar hydroxides with two kinds of metallic cations in the main layers and interlayer domains containing anionic species. This wide family of compounds is sometimes also referred to as anionic clays, by comparison with the more usual cationic clays whose interlamellar domains contain cationic species. LDHs have also been reported as hydrotalcite-like compounds by reference to one of the polytypes of the corresponding [Mg—Al] based mineral. (See “Layered Double Hydroxides: Present and Future”, ed, V Rives, 2001 pub. Nova Science).

By mixed metal compound, it is meant that the atomic structure of the compound includes the cations of at least two different metals distributed uniformly throughout its structure. The term mixed metal compound does not include mixtures of crystals of two salts, where each crystal type only includes one metal cation. Mixed metal compounds are typically the result of coprecipitation from solution of different single metal compounds in contrast to a simple solid physical mixture of two different single metal salts. Mixed metal compounds as used herein include compounds of the same metal type but with the metal in two different valence states e.g. Fe(II) and Fe(III) as well as compounds containing more than two different metal types in one compound.

Classes of inorganic solid mixed metal compounds, which function as phosphate binders, are disclosed in WO 99/15189. For example, mixed metal compounds which are substantially free from aluminum and which have a phosphate binding capacity of at least 30% by weight of the total weight of phosphate present, over a pH range of from 2-8, as measured by the phosphate binding test as described therein. In embodiments, such mixed metal compounds can include iron (Ill) and at least one of magnesium, calcium, lanthanum and cerium. In embodiments, the mixed metal compound can include at least one of hydroxyl and carbonate anions and optionally additionally, at least one of sulphate, chloride and oxide. In one type of embodiment, the mixed metal compound is free of or substantially free of calcium. In embodiments, the mixed metal compound can be a mixed metal hydroxy carbonates containing each of magnesium and iron and be of a hydrotalcite structure. In embodiments, an unaged hydrotalcite can be used. The inorganic solids are water insoluble and can be for oral administration.

Mixed metal compounds for use in the methods disclosed herein can be water insoluble phosphate binders. By water-insoluble phosphate binder, it is meant that the phosphate binder has a solubility in distilled water at 25° C. of 0.5 g/liter or less, or 0.1 g/liter or less, or 0.05 g/liter or less.

The mixed metal compound may also comprise amorphous (non-crystalline) material. By the term amorphous is meant either crystalline phases, which have crystallite sizes below the detection limits of x-ray diffraction techniques, or crystalline phases which have some degree of ordering, but which do not exhibit a crystalline diffraction pattern and/or true amorphous materials which exhibit short range order, but no long-range order.

Because of their water-insolubility, it is preferred if the inorganic mixed metal compounds are in a finely divided particulate form such that an adequate surface area is provided, e.g. over which phosphate binding or immobilization can take place. The inorganic mixed metal compound particles can have a weight median particle diameter (d₅₀) of from 1 to 20 micrometers, or from 2 to 11 micrometers, for example. The inorganic mixed metal compound particles can have a d₉₀ (i.e. 90% by weight of the particles have a diameter less than the d₉₀ value) of 100 micrometers or less, for example.

As described in detail below, mixed metal compounds suitable for use in the methods of the disclosure can be compounds of formula (I), heat-treated compounds of formula (II), and/or bivalent metal depleted compounds of formula (III)-(VII).

In any of the foregoing embodiments, in any of the formulas herein, changing the molar ratio of bivalent to trivalent metal can result in different compositions. For example, by changing the molar ratio of M^{II}:M^{III} cations to 1:1, 2:1, 3:1, 4:1 different composition materials can be achieved.

In any of the embodiments herein, in ant of the formulas herein, the bivalent metal, M^{II}, can be selected from one or more of Mg (II), Zn (II), Fe (II), Cu (II), Ca (II), La (II) and Ni(II). In one class of embodiments, M^{II} includes Mg (II). In embodiments, the compound of formula (I) can be free or substantially free of calcium.

In embodiments, in any of the formulas disclosed herein Aⁿ⁻ can be at least one n-valent anion. The anions Aⁿ⁻ may be selected such that the requirement that compound be charge neutral is satisfied. Aⁿ⁻ can be at least one anion selected from carbonate, hydroxycarbonate, oxo-anions (e.g. nitrates, sulphate), metal-complex anion (e.g. ferrocyanide), polyoxo-metalates, organic anions, halide, hydroxide and mixtures thereof. In embodiments, the anion is carbonate. In embodiments, the n-valent anion Aⁿ⁻ is an exchangeable anion thereby facilitating the exchange of the phosphate for the Aⁿ⁻ valent anion in the solid mixed metal compound.

In embodiments, in any of the formulas disclosed herein, the trivalent metal M^{III} can be selected from one or more of Mn(III), Fe(III), La(III), Ni (III) and Ce(III). Of these, Fe(III) is particularly contemplated. Herein, (II) means a metal in a bivalent state and (III) means a metal in a trivalent state.

In embodiments, the compound contains iron(III) and at least one of Magnesium, Calcium, Lanthanum or Cerium, or at least one of Magnesium, Lanthanum or Cerium, or Magnesium.

In embodiments, M^{II} can be at least one of magnesium, calcium, lanthanum and cerium; M^{III} can be at least iron(III); Aⁿ⁻ is at least one n-valent anion; x=Σny; 0<x≤0.67, 0<y≤1, and/or 0≤z≤10.

In embodiments, the compound can comprise less than 200 g/kg of aluminum, or less than 100 g/kg, or less than 50 g/kg expressed as weight of aluminum metal per weight of compound.

In embodiments, only low levels of aluminum are present, such as less than 10 g/kg, or less than 5 g/kg.

In additional embodiments, the compound is free from aluminum (Al). By the term “free from aluminum” it is meant that the material termed “free from aluminum” comprises less than 1 g/kg, or less than 500 mg/kg, or less than 200 mg/kg, or less than 120 mg/kg expressed as weight of elemental aluminum per weight of compound.

In embodiments, the compound comprises less than 100 g/kg of calcium, or less than 50 g/kg, or less than 25 g/kg expressed as weight of elemental calcium per weight of compound.

In embodiments, only low levels of calcium are present such as less than 10 g/kg, or less than 5 g/kg.

In other embodiments, the compound is free from calcium. By the term “free from calcium” it is meant that the material termed “free from calcium” comprises less than 1 g/kg, or less than 500 mg/kg, or less than 200 mg/kg, or less than 120 mg/kg expressed as weight of elemental calcium per weight of material.

In embodiments, the compound is free from calcium and free from aluminum.

Any of the compounds disclosed herein can be used for one or more of the methods described herein. In embodiments, the compound can be for use as a medicament. In embodiments, the compound can be used for a medicament for binding phosphate. In embodiments, the compound can be used for preventing vascular calcification, reducing vascular calcification, lowering serum PTH, or presenting a rise in serum PTH, and optionally together with prophylaxis or treatment of any one or more of hyperphosphataemia, metabolic bone disease, metabolic syndrome, renal insufficiency, hypoparathyroidism, pseudohypoparathyroidism, acute untreated acromegaly, chronic kidney disease (CKD), clinically significant change in bone mineralization (osteomalecia, adynamic bone disease, osteitis fibrosa), soft tissue calcification, cardiovascular disease associated with high phosphates, secondary hyperparathyroidism, over medication of phosphate salts and other conditions requiring control of phosphate absorption. In embodiments, any of the compounds defined here in can be used in the manufacture of a medicament for the prophylaxis or treatment of any one of hyperphosphataemia, renal insufficiency, hypoparathyroidism, pseudo hypoparathyroidism, acute untreated acromegaly, chronic kidney disease and over medication of phosphate salts.

### Mixed Metal Compounds of Formula I

In embodiments, the solid mixed metal compound can be of formula (I):

M^{II}₁₋ₓ.M^{III}ₓ(OH)₂Aⁿ⁻_{y}.zH₂O, (I)

where M^{II} is at least one bivalent metal; M^{III} is at least one trivalent metal; Aⁿ⁻ is at least one n-valent anion. It will be understood that x=[M^{III}]/[M^{II}]+[M^{III}]) where [M^{II}] is the number of moles of M^{II} per mole of compound of formula I and [M^{III}] is the number of moles of M^{III} per mole of compound of formula I. In embodiments, x=Σny, and x, y and z fulfill 0<x≤0.67, 0<y≤1, and 0≤z≤10.

In the above formula (I), when A represents more than one anion, the valency (n) of each may vary. “Σny” means the sum of the number of moles of each anion multiplied by its respective valency.

In one class of embodiments, 0.1<x, such as 0.2<x, 0.3<x, 0.4<x, or 0.5<x. In an embodiment 0<x≤0.5. In additional embodiments 0<y≤1, 0<y≤0.8, 0<y≤0.6, 0<y≤0.4, 0.05<y≤0.3, 0.05<y≤0.2, 0.1<y≤0.2, or 0.15<y≤0.2.

In embodiments 0≤z≤10, 0≤z≤8, 0≤z≤6, 0≤z≤4, 0≤z≤2, 0.1≤z≤2, 0.5≤z≤2, 1≤z≤2, 1≤z≤1.5, 1≤z≤1.4, 1.2≤z≤1.4, or z is approximately 1.4.

In an embodiment 0<x≤0.5, 0<y≤1, and 0≤z≤10.

It will be appreciated that each of the values of x, y and z described herein may be combined. Thus any combination of each of the values listed in the table below are specifically disclosed herein and corresponding mixed metal compounds are contemplated for the uses and compositions described herein.

| x | y | z |
|---|---|---|
| 0.1 < x | 0 < y ≤ 0.8 | 0 ≤ z ≤ 10 |
| 0.2 < x | 0 < y ≤ 0.6 | 0 ≤ z ≤ 8 |
| 0.3 < x | 0 < y ≤ 0.4 | 0 ≤ z ≤ 6 |
| 0.4 < x | 0.05 < y ≤ 0.3 | 0 ≤ z ≤ 4 |
| 0.5 < x | 0.05 < y ≤ 0.2 | 0 ≤ z ≤ 2 |
| 0 < x ≤ 0.67 | 0.1 < y ≤ 0.2 | 0.15 z 5_2 |
| 0 < x ≤ 0.5 | 0.15 < y ≤ 0.2 | 0.5 ≤ z ≤ 2 |
| | | 1 ≤ z ≤ 2 |
| | | 1 ≤ z ≤ 1.5 |
| | | 1 ≤ z ≤ 1.4 |
| | | 1.1 ≤ z ≤ 1.4 |

The methods of the disclosure can include administering a mixed metal compound of formula (II). Mixed Metal Compounds of Formula (II)

Mixed metal compounds of formula (II) can be prepared by heat treatment of a compound of formula (I).

A solid mixed metal compound of formula (II) can have the following formula:

M^{II}₁₋ₐ.M^{III}ₐO_{b}Aⁿ⁻_{c}.zH₂O (II)

where M^{II} is at least one bivalent metal (i.e. with two positive charges); M^{III} is at least one trivalent metal (i.e. with three positive charges); Aₙ is at least one n-valent anion; 2+a=2b+Σcn; a=number of moles of M^{III}(number of moles of M^{II}+number of moles of M^{III}); and Σcn<0.9a.

In the above formula (II), when A represents more than one anion, the valency (i.e. the charge of the anion) (n) of each may vary. In the above formula (Ii), “Σcn” means the sum of the number of moles of each anion, per mole of compound of formula (II), multiplied by its respective valency.

In embodiments, the value of z is suitably 2 or less, 1.8 or less, 1.5 or less. In embodiments, value of z may be 1 or less.

In embodiments, a is from 0.1 to 0.5, from 0.2 to 0.4. In embodiments, the value of b is 1.5 or less, or 1.2 or less. In embodiments, the value of b is greater than 0.2, more greater than 0.4, greater than 0.6, or greater than 0.9,

In embodiments, when a is >0.3 it is preferred that Σcn<0.5a. When a is ≤0.3 it is preferred that Σcn<0.7a.

The value of c for each anion is determined by the need for charge neutrality as expressed by the formula 2+a=2b+Σcn.

### Bivalent Metal Depleted Mixed Metal Compounds

Mixed metal compounds can also be depleted of bivalent metals by chemical treatment, as described in more detail below.

In embodiments, such a mixed metal compound can be a compound of formula (III):

M^{II}₁₋ₐM^{III}ₐ (III)

wherein M^{II} is at least one bivalent metal; M^{III} is at least one trivalent metal; and 1>a>0.4; the compound contains at least one n-valent anion Aⁿ⁻ such that the compound is charge neutral.

In embodiments, the mixed metal compound having reduced bivalent metal content can be obtained or obtainable by treatment of a compound of formula (IV) with an acid, a chelating agent or a mixture thereof of a formula (IV)

[M^{II}₁₋ₐM^{III}ₐO_{b}(OH)_{d}](Aⁿ⁻)_{c}.zH₂O (IV)

wherein M^{II} is at least one bivalent metal; M^{III} is at least one trivalent metal; and 0<a≤0.4; the compound contains at least one n-valent anion Aⁿ⁻ such that the compound is charge neutral. In embodiments, M^{II} is at least one bivalent metal selected from Mg (II), Zn (II), Fe (II), Cu (II), Ca(II), La (II); M^{III} is at least one trivalent metal selected from Mn(III), Fe(III), La(III) and Ce(III); and Aⁿ⁻ is at least one n-valent anion and wherein at least one anion is carbonate; 0<a<0.4; 0<b≤2. The value of c for each anion is determined by the need for charge neutrality as expressed by the formula 2+a−2b−d−cn=0; and 0<d≤2, and 0<z≤5.

The result of contacting a compound of formula (IV) with an acid, a chelating agent, or a mix thereof can be a compound of formula (V)

[M^{II}₁₋ₐM^{III}ₐO_{b}(OH)_{d}](Aⁿ⁻)_{c}.zH₂O (V)

wherein M^{II} is at least one bivalent metal; M^{III} is at least one trivalent metal; and 1>a>0.4; the compound contains at least one n-valent anion Aⁿ⁻ such that the compound is charge neutral. In embodiments, a in formula (IV) is 1>a>0.4, 0<b≤2, 0<d≤2, 0<z≤5. The value of c for each anion is determined by the need for charge neutrality as expressed by the formula 2+a−2b−d−cn=0.

In embodiments, 0<d≤2. In embodiments, d is 1.5 or less, or d is 1 or less. In embodiments 0<d≤1, or 0≤d≤1.

In embodiments, d is 0 and the compound is thus a compound of formula (VI). When d is 0, optionally Σcn<0.9a.

M^{II}₁₋ₐM^{III}ₐO_{b}(Aⁿ⁻)_{c}.zH₂O (VI)

wherein M^{II} is at least one bivalent metal; M^{III} is at least one trivalent metal; and 1>a>0.4; the compound contains at least one n-valent anion Aⁿ⁻ such that the compound is charge neutral. In embodiments, a in formula (IV) is 1>a>0.4, 0<b≤2, 0<z≤5. The value of c for each anion is determined by the need for charge neutrality as expressed by the formula 2+a−2b−d−cn=0.

In embodiments, 0<b≤2, or 1.5 or less, 1.2 or less, or 1 or less. In embodiments 0<b≤1.5, or 0≤b≤1.5, or 0<b≤1.2, or 0≤b≤1.2, or 0<b≤1, or 0≤b≤1.

In embodiments, b is 0 and the compound is thus a compound of formula (VII):

M^{II}₁₋ₐM^{III}ₐ(OH)_{d}](Aⁿ⁻)_{c}.zH₂O (VII)

wherein M^{II} is at least one bivalent metal; M^{III} is at least one trivalent metal; and 1>a>0.4; the compound contains at least one n-valent anion Aⁿ⁻ such that the compound is charge neutral. In embodiments, in formula (VII) 2+a−d−cn=0; Σcn<0.9a, 0≤d<2, and 0<z≤5.

If b is not 0, optionally c can be 0.5 or 0.15 or less. In embodiments, in any of the foregoing formulas of a bivalent metal depleted compound, 0<c≤0.5, or 0<c≤0.15, or 0≤c≤0.15, or 0.01<c≤0.15, or 0.01≤c≤0.15.

In embodiments, in any of the formulas disclosed herein M^{II} can be at least one bivalent metal selected from Mg (II), Zn (II), Fe (II), Cu (II), Ca(II), La(II), Ce (II) and Ni(II). In embodiments, M^{III} can be at least one trivalent metal selected from Mn(III), Fe(III), La(III) and Ce(III). M^{II} and M^{III}—can be different metals or they can be the same metals but in different valence states. For instance, M^{II} may be Fe(II) and M^{III} may be Fe(III). M^{III} may be Al(III) for treatments where aluminum accumulation and toxic complications are not a problem. In embodiments, the compound is substantially or totally free of aluminum.

In embodiments, Fe(III) can be used as the trivalent metal. In bivalent metal depleted compounds, Fe(III) does not dissolve simultaneously with the Mg(II) during the depletion process thereby enabling the formation of a Mg-depleted compound. In contrast, mixed metal compounds prepared from Mg Al are more difficult to deplete because of a more similar dissolution profile of the Mg and Al metal resulting in compounds of more equimolar ratios.

In embodiments, in any of the foregoing formulas of a bivalent metal depleted compound, 0<z≤5, or 0<z≤2, or 0≤z≤2, or 0<z≤1.8, or 0≤z≤1.8, or 0<z≤1.5, or 0≤z≤1.5.

In embodiments, in any of the foregoing formulas of a bivalent metal depleted compound, such as in formulas (III), (V), (VI), and (VII), a may be any value between 1 and 0.4. Thus 1>a>0.4. In embodiments, 0.98>a>0.5, 0.98>a>0.6, 0.98>a≥0.7, 0.95>a≥0.7, 0.90>a≥0.7, 0.85>a≥0.7, 0.80>a≥0.7.

The increase of the value of “a” above 0.98 results in more significant reduction in phosphate binding of up to 75%. Without being bound by theory it is believed that the decreased phosphate binding for values of “a” above 0.98 results from the complete removal of the bivalent metal (e.g. magnesium); furthermore, the yield (the amount of phosphate binder isolated after the depletion-reaction) is reduced significantly because of loss of the iron. This makes the compound structurally unstable and thereby less effective as a phosphate binder. Whereas if the value of “a” is 0.98>a≥0.7 phosphate binding may be reduced by only approximately 10%. If the value of “a” is below 0.7 phosphate binding is either higher or maintained. If the “a” value is above 0.8 the potential for release of the bivalent metal (magnesium) is still more than 50% of the total available amount of bivalent metal present in un-depleted phosphate binder thereby providing the potential undesirable release of metal. Consequently a contemplated range is between 0.80>a≥0.7 as this provides the best compromise between good phosphate binding and lower amounts of bivalent metal available for dissolution. Coincidentally, this also falls within the pH region of 4-6 whereby the largest pH buffering is observed of the undepleted material and where a transformation from the presence of a crystalline (hydrotalcite) to a non-crystalline structure is observed. Typically, the yield of the depletion reaction is not less than 50% if a≥0.7.

In addition, depleted compounds of “a” values above 0.95 are more difficult to consistently manufacture and phosphate binding is reduced and approaches that of a sample of FeOOH (“a” value is 1). Pure FeOOH compounds are less stable and require the presence of a stabilizing agent e.g. carbohydrate. For values of “a” obtainable from the compounds isolated from a solution maintained at pH values of 8, 9 or higher, phosphate binding occurs mainly only through ion-exchange of the phosphate anion in solution with the anion present in the solid layered double hydroxide or mixed metal compound. The maximum phosphate binding capacity of the layered double hydroxides structure or the mixed metal compounds with values of “a” below 0.4 are then limited by the amount of the exchangeable anion and its associated charge within the starting material, in addition, the available size of the space between the layers of the mixed metal compound is also restricting the exchange of phosphate at “a” values below 0.4. Values of “a” above 0.4 are known to those skilled in the art to lead to less stable layered double hydroxide structures and these compositions have therefore previously not been considered as effective binders of anions such as phosphate. Despite the gradual loss of the typical layered double hydroxide or hydrotalcite structure, phosphate binding actually increases or is typically maintained at values of “a” above that of 0.4 and only decreases significantly when “a” is above 0.98. It is believed that the higher amount of the trivalent metal maintains good phosphate binding because of a higher net positive charge on the metal hydroxide layers compared to samples with less of the trivalent metal but without the restrictions in phosphate binding observed for those compounds of “a” values below 0.4. Moreover, single metal trivalent metal hydroxide such as ferric hydroxides or ferric citrate compounds are less effective phosphate binders showing that the presence of some bivalent metal is preferred but not at levels resulting in ratios of mixed metal compounds of those of “a” values below 0.4. In addition, simple mixtures prepared from mixtures of magnesium and iron salts are not as effective.

In effect because of exposure of the mixed metal compounds to a depleting agent, prior to use as a medicament, release of solubilized metal can be reduced upon subsequent further contact with gastric acid in the stomach, while maintaining good phosphate binding activity in the gut. The degree of reduction in the bivalent metal can be tailored to any given degree, e.g. from a slight reduction to a significant reduction.

In embodiments, the solid mixed metal compound comprises at least some material, which is a Layered Double Hydroxide (LDH). More preferably, the mixed metal compound of formula (I) is a layered double hydroxide. As used herein, the term “Layered Double Hydroxide” is used to designate synthetic or natural lamellar hydroxides with two different kinds of metallic cations in the main layers and interlayer domains containing anionic species. This wide family of compounds is sometimes also referred to as anionic clays, by comparison with the more usual cationic clays whose interlamellar domains contain cationic species. LDHs have also been reported as hydrotalcite-like compounds by reference to one of the polytypes of the corresponding [Mg—Al] based mineral.

In embodiments, mixed metal compound contains at least one of carbonate ions, and hydroxyl ions.

In embodiments compound contains as M^{II} and M^{II}, magnesium and iron (III) respectively.

The solid mixed metal compound or compounds may be suitably made by co-precipitation from a solution, e.g. as described in WO 99/15189, followed by centrifugation or filtration, then drying, milling and sieving. Alternatively, mixed metal compound may be formed by heating an intimate mixture of finely divided single metal salts at a temperature whereby solid-solid reaction can occur, leading to mixed metal compound formation.

The solid mixed metal compound of formula (I) may be calcined by heating at temperatures in excess of 200° C. in order to decrease the value of z in the formula.

In embodiments, the compound of formula I is formed with no aging or hydrothermal treatment to avoid the crystals of the compound growing in size and to maintain a high surface area over which phosphate binding can take place. The unaged compound of formula I is also optionally maintained in a fine particle size form during the post-synthesis route to maintain good phosphate binding.

In embodiments, a mixed metal compound can include at least Mg²⁺ and at least Fe³⁺, wherein the molar ratio of Mg²⁺ to Fe³⁺ is 2.5:1 to 1.5:1, the mixed metal compound has an aluminum content of less than 10000 ppm, the average crystal size of the mixed metal compound is from 10 to 20 nm (100 to 200 Δ), and the interlayer sulphate content of the compound is from 1.8 to 5 wt % (such as from 1.8 to 3.2 wt %). In embodiments, a mixed metal compound can include at least Mg²⁺ and at least Fe³⁺, wherein the molar ratio of Mg²⁺ to Fe³⁺ is 1.5:1 to 2.5:1, the mixed metal compound has an aluminum content of less than 10000 ppm, the average crystal size of the mixed metal compound is from 10 to 20 nm (100 to 200 Å), and the d50 average particle size of the mixed metal compound is less than 300 μm.

The mixed metal compound can have a dry solid content of at least 10 wt %, or at least 15 wt %, or at least 20 wt %.

When dried, the mixed metal compound has a dry solid content of at least 80 wt %, or more than 85 wt %. The dried mixed metal compound can have a dry solid content of less than 99 wt %, or less than 95 wt %. The dried mixed metal compound can have a dry solid content from 90 to 95 wt %.

As discussed herein, the compound can have an average crystal size of less than 20 nm (200 Å). In embodiments, the compound has an average crystal size of from 100 to 200 Å, 155 to 200 Δ, 110 to 195 Δ, 110 to 185 Å, 115 to 165 Å, 120 to 185 Å, 130 to 185 Å, 140 to 185 Å, 150 to 185 Å, 150 to 175 Å, 155 to 175 Å, 155 to 165 Å.

### Methods of Making Compounds of Formula (I)

In embodiments, the mixed metal compound can be formed by the reaction of an aqueous mixture of magnesium sulphate and ferric sulphate with an aqueous mixture of sodium hydroxide and sodium carbonate, for example. The precipitation can be carried out at a pH of around 9.8 and a reaction temperature starting at around 22° C. and rising to up to 30° C. upon addition of reactants. The resulting precipitate is filtered, washed, dried and milled. The synthesis reaction is represented thus:

4MgSO₄+Fe₂(SO₄)₃+12NaOH+(XS+1)Na₂CO₃→Mg₄Fe₂(OH)₁₂.CO₃.nH₂O+7Na₂SO₄+XSNa₂CO₃.

This generates a mixed metal compound with a molar ratio of Mg:Fe of typically 2:1 and the reaction by-product sodium sulphate. Excess (XS) sodium carbonate added to the reaction mixture along with the sodium sulphate is washed out of the precipitate.

### Method of Making Compounds of Formula (II)

In an embodiment, the compound is a compound of formula (I) in which M^{II} is one or more bivalent metals and is at least Mg²⁺; M^{III} is one or more trivalent metals and is at least Fe³⁺; Aⁿ⁻ is one or more n-valent anions and is at least CO₃²⁻; and 1.0<x/Σyn<1.2, 0<x≤0.67, 0<y≤1 and 0<m≤10.

The method by which the molecular formula of a mixed metal compound may be determined will be well known to one skilled in the art. It will be understood that the molecular formula may be determined from the analysis of M^{II}/M^{III} ratio (Test Method 1), SO₄ analysis (Test Method 5), CO₃ analysis (Test Method 6) and H₂O analysis (Test Method 10).

In embodiments 0<x≤0.4, 0<y≤1 and 0<m≤10.

In embodiments, 1.05<x/Σyn<1.2, 1.05<x/Σyn<1.15, or x/Σyn=1.

In embodiments, 0≤z≤10, 0≤z≤8, 0≤z≤6, 0≤z≤4, 0≤z≤2, 0≤z≤1, 0≤z≤0.7, 0≤z≤0.6, 0.1≤z≤0.6, 0≤z≤0.5, 0≤z≤0.3, 0≤z≤0.15, or 0.15≤z≤0.5 The number of water molecules m can include the amount of water that may be absorbed on the surface of the crystallites as well as interlayer water. The number of water molecules is estimated to be related to x according to: z=0.81−x.

It will be appreciated that each of the preferred values of x, y, z and m may be combined.

In embodiments, the compound has an aluminum content of less than 5000 ppm, or less than 1000 ppm, or about 100 ppm, or about 30 ppm.

In embodiments, the total sulphate content of the compound is from 1.8 to 5 wt %. By total sulphate content it is meant content of sulphate that is present in the compound. This may be determined by well-known methods, for example, in accordance with Test Method 1. In embodiments, the total sulphate is from 2 to 5 wt %, 2 to 3.7 wt %, 2 to 5 wt %, 2 to less than 5 wt %, 2.1 to 5 wt %, 2.1 to less than 5 wt %, 2.2 to 5 wt %, 2.2 to less than 5 wt %, 2.3-5 wt %, or 2.3 to less than 5 wt %.

In embodiments, the total sulphate content of the compound can be from 1.8 to 4.2 wt %, 2 to 4.2 wt %, 2 to 3.7 wt %, 2 to 3.2 wt %, 2 to less than 3.2 wt %, 2.1 to 3.2 wt %, 2.1 to less than 3.2 wt %, 2.2 to 3.2 wt %, 2.2 to less than 3.2 wt %, 2.3-3.2 wt %, or 2.3 to less than 3.2 wt %.

The compound will also contain an amount of sulphate that is bound within the compound. This content of sulphate, the interlayer sulphate, may not be removed by a washing process with water. As used herein, amounts of interlayer sulphate are the amount of sulphate as determined in accordance with Test Method 5. In embodiments, the interlayer sulphate content of the compound can be from 1.8 to 5 wt %, 1.8 to 3.2 wt %, 2 to 5 wt %, 2 to less than 5 wt %, 2 to 3.2 wt %, 2 to 3.1 wt %, 2 to 3.0 wt %, 2.1 to 5 wt %, 2.1 to 3.2 wt %, 2.1 to less than 3.2 wt %, 2.2 to 5 wt %, 2.2 to 3.2 wt, 2.2 to less than 3.2 wt %, 2.3 to 5 wt %, 2.3 to 3.2 wt %, 2.3 to less than 3.2 wt %, 2.5 to 5 wt %, 2.5 to 3.2 wt %, 2.5 to less than 3.2 wt %, and 2.5 to 3.0 wt %.

A mixed metal compound in embodiments can comprising at least Mg²⁺ and at least Fe³⁺, the molar ratio of Mg²⁺ to Fe³⁺ can be 2.5:1 to 1.5:1, the mixed metal compound can have an aluminum content of less than 10000 ppm, the average crystal size of the mixed metal compound can be from 10 to 20 nm (100 to 200 Å), and the d50 average particle size of the mixed metal compound can be less than 300 μm. In embodiments, the d50 average particle size of the mixed metal compound is less than 200 μm.

In embodiments, the mixed metal compound can have a water pore volume of from 0.25 to 0.7 cm³/g of mixed metal compound, 0.3 to 0.65 cm³/g of mixed metal compound, 0.35 to 0.65 cm³/g of mixed metal compound, or 0.3 to 0.6 cm³/g of mixed metal compound.

In embodiments, the nitrogen pore volume of the mixed metal compound can be from 0.28 to 0.56 cm³/g. As used herein, the term ‘nitrogen pore volume’ refers to the pore volume as determined in accordance with Test Method 14. When the nitrogen pore volume of the mixed metal compound is from 0.28 to 0.56 cm³/g the close correlation to the water pore volume is such that the water pore volume need not be determined.

In embodiments, the mixed metal compound has a surface area is from 80 to 145 m² per gram of compound. In alternative embodiments, the mixed metal compound has a surface area from 40 to 80 m² per gram of compound.

In embodiments, the d50 average particle size of the mixed metal compound is less than 100 μm, less than 50 μm, less than 20 μm, less than 10 μm. In embodiments, the d50 average particle size of the mixed metal compound is approximately 5 μm.

In one type of embodiment, the mixed metal compound can be a calcined mixed metal compound. Such calcined mixed metal compounds are described in further detail below. The release of the bivalent metal, e.g. magnesium, associated with the pharmaceutical use of compounds of WO-A-99/15189 can be reduced by heat treatment of a suitable mixed metal compound, for example a layered double hydroxide or a compound having a hydrotalcite structure. It can similarly reduce the release of other bivalent metals when M″ is other than magnesium.

The process for preparing compounds of formula (II) results in changes in the structural detail of the compound which is the starting material. Therefore, the formula (II) as written is only intended to describe its elemental composition and should not be taken as a definition of structure.

When the compound of formula (II) comprises magnesium as M^{II} and iron as M^{III} cations and carbonate as an anion, preferably it exhibits an x-ray diffraction peak at 34° 2Θ. At lower temperatures (≤250° C.), conflicting peaks from the layered double hydroxide may be present whereas when the temperature rises (>400° C.), a conflicting peak due to the oxide M¹¹O may appear but these peaks may be resolved using deconvolution methods.

In embodiments a solid mixed metal compound of formula (II) can be obtained by or obtainable by heating at a temperature of in a range of 200° C. to 600° C., or in a range of 225° C. to 550° C., or in a range of 250° C. to 500° C. of a compound of formula (I):

M^{II}₁₋ₓ.M^{III}ₓ(OH)₂Aⁿ⁻_{y}.zH₂O, (I)

where M^{II} is at least one bivalent metal; M^{III} is at least one trivalent metal; Aⁿ⁻ is at least one n-valent anion. It will be understood that x=[M^{III}]/[M^{II}]+[M^{III}]) where [M^{II}] is the number of moles of M^{II} per mole of compound of formula I and [M^{III}] is the number of moles of M^{III} per mole of compound of formula I. In embodiments, x=Σny, and x, y and z fulfill 0<x≤0.67, 0<y≤1, and 0≤z≤10.

It should be noted that formula (I) is to be interpreted in such a way as to preserve overall charge neutrality and can include any variations described above. In formula (I) and/or formula (II) subclasses of compounds of either formula may comprise, respectively, those wherein x or a is less than any of the following values and those wherein x or a is greater than or equal to any of those values, these values being 0.1, 0.15, 0.2, 0.25, 0.3, 0.35, 0.4, 0.45. One such example comprises the subclasses, wherein a is, respectively, greater than or equal to 0.3, and less than 0.3. The value of x is suitably from 0.1 to 0.5, or from 0.2 to 0.4. In formula (I), Σny is the sum of the number of each anion multiplied by its respective valency.

In embodiments, the mixed metal compound can be made by heat treatment of a suitable starting material of formula (I) as hereinbefore defined. Optionally other preparation methods may be employed to prepare the mixed metal compound such as solid state synthesis, solid-solid reactions or highly intensively milling of single or mixed metal oxides or hydroxides using hydrothermal routes or low temperature routes.

The mixed metal compound of formula (II) can be prepared by heat treatment of a suitable starting material of formula (I) as hereinbefore defined may be prepared by providing a first solution of a water soluble compound of metal M^{II} and a water soluble compound of metal M^{III}, the anions being chosen so as not to result in precipitation from the first solution. A second solution is also provided, of a water soluble hydroxide (e.g. NaOH) and a water soluble salt of anion Aⁿ (the cation being chosen so as not to precipitate with the hydroxide or the anion with the metal from the hydroxide). The two solutions are then admixed and the mixed metal compound starting material is formed by co-precipitation. It comprises solid crystalline material, usually also with presence of some solid amorphous material. Preferably, at least some of the material so formed is of a layered double hydroxide and/or of a hydrotalcite structure, usually also with some amorphous and/or poorly crystalline material, preferably after co-precipitation, the material is then filtered or centrifuged, washed then dried by heating.

In embodiments, the material is washed in order to remove the water-soluble salts that are the by-product of the precipitation reaction. If significant amounts of these soluble salts are left admixed with the solid precipitate, then the subsequent heating of the material may result in the incorporation of the soluble salts into the resulting solid, potentially having an adverse effect on its phosphate binding behavior. The material can be washed such that the remaining level of water soluble salts (having a solubility in water of 1 g/liter or more) is less than 15%, or less than 10%, or less than 5% by weight of the solid mixed metal compound after drying as described below.

After the filtering or centrifuging and washing, the drying is optionally carried out at low temperature (such as up to 120° C.), for example by oven drying, spray drying or fluid bed drying.

Optionally, the dry material may be treated prior to heat treatment, to remove oversize particles by milling and/or sieving and/or any other suitable technique, for example to restrict the material to be heat treated to particles which are substantially no greater than 100 μm in diameter. Preferably, as measured by sieving, less than 10% by weight of particles are greater than 106 μm in diameter, or less than 5%. In one type of embodiment, no particles are greater than 106 μm in diameter as measured by sieving. The resultant dry material is then directly subjected to the necessary heat treatment, e.g. at a temperature of at least 200° C. or in a range of 225° C. to 550° C., or in a range of 250° C. to 500° C., for example by means of oven drying or drying in a rotary calcinator or fluid bed dryer. Optionally, the wet cake material may be directly subjected to temperatures above 200° C. without low temperature drying (such as up to 120° C.) and milling.

The heating can results in a reduction in the amount of loss into solution of metal M^{II} from the heat-treated compound by at least 5% by weight, or 10% by weight, or 15% by weight, or 20% by weight, or 25% by weight, or 30% by weight, or 35% by weight, or 40% by weight, or 45% by weight, or 50% by weight compared to loss from the untreated compound, when measuring the loss of metal M″ using the test as hereinafter described.

The substances of the disclosure may contain at least one compound of formula (I) but the process mentioned above for making the starting material may also cause other materials to be present in the intermediate product e.g. of formula (II) and in the final product, for example single (as opposed to mixed) metal compounds which may also be formed during the co-precipitation process.

The heating can be at a temperature in a range of 200° C. to 600° C., or 225° C. to 550° C., or 250° C. to 500° C. In embodiments, this can result in a reduction in the amount of metal M^{II} lost to solution by at least 50% by weight compared to that lost from the unheated compound of formula (I), under the conditions described in more detail herein. If less reduction in the amount of loss into solution of metal M^{II} from the heat-treated compound is desired, then the temperature is suitably lower, and can be lower than 200° C. in embodiments.

The heating can be carried out in a heated environment in a range of 200° C. to 600° C., or 225° C. to 550° C., or 250° C. to 500° C. for a period of 1 minute or longer, or 5 minutes or longer, or 1 hour or longer. The compound can be in the heated environment for 10 hours or less, or 5 hours or less, or 3 hours or less. If less reduction in the amount of loss into solution of metal M^{II} from the heat-treated compound is desired, then the time is suitably shorter and can be less than 1 minute in embodiments.

The heating as described above results in the calcination of the compound according to formula (I). The calcination is believed to lead to the formation of a substance according to formula (II). This results in the value of a for a compound according to formula (II) being less than or equal to the value of x for the corresponding untreated compound according to formula (I). The calcination is preferably not excessive in terms of temperature and/or time of calcination, by which it is meant that the calcination temperature should not exceed 600° C. for more than 3 hours, otherwise a phosphate binding performance which is less than optimal may be found.

Excessive calcination results in the reduction of the value of Σcn/a from formula (II) to less than 0.03. Hence it is contemplated that Σcn/a can be greater than 0.03, or greater than 0.05, or greater than 0.09, or greater than 0.10. Excessive calcination also may lead to the formation of a Spinel crystalline structure, hence it is preferred that the substances of the disclosure do not exhibit a Spinel structure by x-ray diffraction. Spinel has a value for a of 0.67 and so it is preferred if the compound of formula (II) has a value for a of 0.66 or less, or 0.5 or less, more preferably 0.3 or less.

In one type of embodiment, calcination of the compound of formula (II) can results in a substance with at least a 10% higher phosphate binding capacity relative to that of the compound of formula (I) from which the substance is obtained or obtainable by calcination.

A suitable method for monitoring the degree of calcination is by measurement of the percentage loss of crystalline surface water at 105° C. This is measured by allowing a sample to reach an equilibrium moisture content by storage for several days at ambient conditions (20° C., 20% RH), weighing the sample, then heating at 105° C. for 4 hours and reweighing to establish the loss in weight, expressed as a percentage. Drying at 105° C. removes the surface absorbed water (i.e. non-chemically-bound water or water on the crystal surface)

In embodiments, the mixed metal compound after calcination has less than 2%, or less than 1.5%, or less than 1% by weight crystallite-surface absorbed water.

### Method for Making Bivalent Metal Depleted Mixed Metal Compound

In embodiments, a mixed metal compound obtained by or obtainable by treatment of a compound of formula (I) or a compound of formula (II) with an acid, a chelating agent or a mixture thereof.

In embodiments, a compound of formula (V) can be made by contacting a compound of formula (IV) with an acid, a chelating agent or a mixture thereof; and b) optionally subjecting the resulting compound to heat treatment.

As with the other mixed metal compounds described herein, the compound of formula (III) or (V) can be provided in a pharmaceutical composition comprising the compound of formula (III) or (V) and a pharmaceutically acceptable carrier, diluent, excipient or adjuvant.

In embodiments, a bivalent metal depleted compounds can be provided comprising oxide-hydroxide of metal having a M-O bond distance of approximately 2 θ (angstrom) as determined by Extended X-Ray Absorption Fine Structure (EXAF) studies. More specifically, for depleted compound derived from a Mg Fe mixed metal compound, the distance between the center absorbing iron atom and its nearest oxygen atom neighbor is 1.994 θ (1st shell distance). The distance between the center absorbing iron atom and its nearest iron neighbor (M-O-M distance) is 3.045 θ (2nd shell distance). A contemplated range M-O bond distance is between 1.5-2.5 θ and another range of M-O-M distance is between 2-4 Θ. Under controlled conditions it is possible to remove the more soluble metal from the mixed metal compounds such as layered hydroxide structure or a heat-treated mixed metal compound while maintaining mixed metal compounds with bivalent:trivalent molar ratios less than 1 with a typical hydrotalcite XRD signature, thereby creating metal-depleted mixed metal compounds with, e.g. improved or maintained phosphate binding and a lower release of bivalent or trivalent metal ions (such as magnesium) during the phosphate binding reaction. In addition or alternatively, the metal-depleted mixed metal compound may be heat-treated to increase phosphate-binding and reduce metal (e.g. magnesium) release further. The metal-depleted mixed metal compound has superior phosphate binding characteristics to the mixed metal compounds of WO-A-99/15189, compounds of formula (I) such as described in WO2007/0088343, and formula (II) such as described in WO 2006/085079. The metal-depleted mixed metal compound may be magnesium depleted. The magnesium-depleted mixed metal compound comprises a lower content of the more soluble bivalent magnesium ion and more of the less soluble trivalent iron resulting in ratios of bivalent Mg:trivalent Fe range significantly less than those previously reported for solid mixed metal compounds used for phosphate binding.

In embodiments, carbonate can be used instead of sulphate anion in the starting material, which can aid in obtaining in a cleaner compound i.e. with lower amounts of sulphates salts remaining in the depleted product after acidification of the mixed metal compound; this is because of the acidification of the carbonate anion only leads to formation of water and carbon dioxide.

The substances of the disclosure may contain at least one compound of formula (I) or (IV). The process of preparing bivalent metal depleted compounds such as compounds of formula (III) or (V) may also result in other materials being present in addition to compounds of formula (III) or (V), for example single (as opposed to mixed) metal compounds may also be formed during the process. The process for preparing compounds of formula (III) or (V) may result in changes in the structure of the compound which is the starting material. Therefore, the formula (III) or (V) describe only the elemental composition of compounds of formula (III) or (V) and do not provide a definition of structure.

In embodiments, the compound of formula (III) or (V) can be formed with no aging or hydrothermal treatment to avoid the crystals of the compound growing in size and to maintain a high surface area. In embodiments, the compound of formula III or V can be maintained in a fine particle size form during the post-synthesis route, which can aid in maintaining good phosphate binding. In embodiments, 90% of the compound of formula III or V based on volume (d90) has a particle size of less than 200 micron, more preferably 90% of the compound of formula III or V based on volume (d90) has a particle size of less than 100 micron, most preferably 90% of the compound of formula III or V based on volume (d90) has a particle size of less than 50 micron.

The depleting agent can be selected from HCI, H₂SO₄, citric acid, EDTA, HNO₃, acetic acid and aluminum sulphate [AI₂(SO₄)₃] and combinations hereof. In embodiments, the acid or chelating agent is hydrochloric acid.

The concentration of the depleting agent may range from about 0.01 M to about 5M. In embodiments, the structures are depleted (such as in magnesium) using depleting agent of concentration 0.01 M to 5 M, or a concentration from 0.1 to 2 M, or from 0.5 to 1.5 M.

In embodiments, the process provides a reduction of the amount of metal M^{II} by at least 1% by weight compared to that of the untreated compound of formula (IV), or at least 2% by weight, or at least 3% by weight, or at least 4% by weight, or at least 5% by weight, or at least 6% by weight, or at least 7% by weight, or at least 8% by weight, or at least 9% by weight.

In embodiments, treatment with hydrochloric acid (HCI) can be carried out with HCI of concentration in a range of 0.01 M to 5 M, or in a range of 0.1 to 2 M, or in a range of 0.5 to 1.5 M.

In embodiments, the treatment can be applied for a period of at least 1 minute, or 2 minutes, or 3 minutes, or 4 minutes, or 5 minutes or longer, 15 minutes or longer, 1 hour or longer.

In an embodiment, the compound of formula (IV) wherein 0<a≤0.4 may be treated for 1 hour or less, or 30 minutes or less, or 15 minutes or less.

The optimum in treatment time may vary depending on the conditions of the treatment e.g. amount of starting material, acid concentration, type of acid, treatment pH, desired level of depletion, etc. The treatment time will be shorter when using stronger acids whereas treatment time will increase with weaker acid strengths. Optionally, the acid strength is not too weak (less than 0.1M), as this would increase production time as well as increasing the volume of acid required.

The treatment as described above results in the reduction of the bivalent metal ion from the compound according to formula (IV), or a compound according to formula (I), or a compound according to formula (II). This results in the value of a for the treated compound being equal to or larger than the value of a for the corresponding untreated compound.

The depletion treatment is preferably not excessive in terms of acid and/or chelating agent concentration and/or time of exposure, by which it is meant that the treatment should not exceed treatment for more than 2 hours, otherwise a phosphate binding performance which is less than optimal may be found.

Treatment with acid below pH=3 (i.e. contacting the compound for a sufficient time with acid until an equilibrium pH 3 is reached and then maintaining at the equilibrium value for sufficient time (e.g. a 30 minute time period can be used for the total of the initial addition and for maintaining the pH constant) results in the increase of the value of a to more than 0.98 and significant reduction in phosphate binding. Hence it is preferred that a is less than 0.99, more preferably less than 0.95, even more preferably less than 0.9, most preferably less than 0.85. Excessive treatment with acid may lead to complete dissolution of the compound with significant reduction in phosphate binding performance or yield of preparation, hence it is preferred that the compounds are not completely dissolved.

Treatment with acid at or below pH 5 results in complete loss of the hydrotalcite XRD signal. Without being bound by theory, it is believed that the bivalent metal-depleted compounds obtained at pH of 5 or less are the result of the transition from the crystalline hydrotalcite into a non-crystalline phase. The non-crystalline phase is structurally stable but when obtained at pH values of pH 3 or below will also start releasing the trivalent metal ions. Consequently, there is an optimum pH range to which the material is depleted. Depleted compounds obtained at pH 5 typically have a value for a of not more than 0.85 and so it is contemplated that the compound of formula (III) can have a value for a of 0.85 or less, or 0.8 or less, or not less than 0.4, or not less than 0.5, or not less than 0.6, or not less than 0.7. In certain embodiments, a value of a of not less than 0.7 is preferred because the depleted compound of an a value of 0.7 has approximately a 50% reduction of the release of the bivalent metal into solution during the phosphate binding. Assuming equivalent phosphate binding capacity, an equivalent average daily dose of magnesium-depleted Mg Fe mixed metal compound (i.e. 3 to 4.5 g of example A) containing 50% less magnesium would be expected to increase serum magnesium by between 0.12 and 0.18 mmol/1 whereas an increase of 0.24 and 0.36 mmol/1 would be expected for use of the equivalent compound with no depletion when taken by kidney patients. In contrast, subjects with normal functioning kidneys would not see an increase in serum magnesium when taking either the depleted compound or the un-depleted compound from an average baseline of 0.95 mmol/1. A controlled use of a small amount (e.g., leading to an increase serum magnesium of less than 0.12 mmol/l) of magnesium supplementation or even larger amounts, (e.g. leading to an increase of serum magnesium of more than 0.24 mmol/l) of magnesium supplementation may be of benefit to patients described herein.

In embodiments, a bivalent metal depleted compound of formula (III), (V), (VI) and/or (VII) can have at least a 5% higher phosphate binding capacity when measured according to the standard phosphate binding method (Test Method 11a) or not more than 25% reduction in phosphate binding capacity when measured according to the representative test method (Test Method 11b or method 11c) relative to that of the untreated starting compound from which the bivalent metal depleted compound is obtained or obtainable by treatment with acid or chelating agent.

In an embodiment, a method for monitoring the degree of acid addition is by continuous measurement of the pH with a pH meter (Jenway 3520) using a combined glass electrode (VWR 6621759). The pH meter is calibrated with buffers of pH 4, 7 and 10 before any measurement. The pH of the solution is adjusted using minimum volume of the acid and/or chelating agent solution at room temperatures 20+/−5° Celsius. The total volume added for pH adjustment never exceeds 60% of the total volume.

In an embodiment, a method for monitoring the bivalent metal depletion of the compound is by measurement of the metal oxide content, i.e. where the compound is magnesium depleted by measuring the MgO content. This is measured by XRF (PW2400 Wavelength Dispersive XRF Spectrometer).

In an embodiment, a method for monitoring the bivalent metal depletion of the compound is by measurement of the magnesium (or other bivalent metal) released from the compound during the phosphate binding.

In one type of embodiment, a magnesium-depleted mixed metal compound after treatment can have less than 28%, or less than 25%, or less than 20% but does not have less than 0.5% by weight MgO content.

Phosphate is also believed to bind to the depleted compound through a direct ionic interaction between one or two negatively charged oxygen ions on the phosphate with the M(III) metal center in the solid through displacement of hydroxide. The biggest increase in phosphate binding and/or reduction in magnesium release is for those compounds isolated from solution where the pH is within the pH buffering region of the starting material from which the M(II) depleted material is derived. Depleted compounds isolated at very low pH (pH 3 or less) result in lower phosphate binding, lower yield and also more significant dissolution of the trivalent cation whereas depleted compounds isolated at high pH values 8 or 9 are not sufficiently depleted to improve phosphate binding above that of the starting material or show more release of the bivalent metal.

The increase in phosphate removal by the M(II) depleted compound correlates with the increase in pH buffering capacity of the mixed metal compound from which the M(II) depleted completed compound is derived. Consequently, the presence of hydroxide (OH) groups in the M(II)-depleted compound is preferred for binding phosphate such as of formula: M^{II}ₐM^{III}₁₋ₐ(OH)_{d}, [M^{II}ₐM^{III}₁₋ₐ(OH)_{d}](Aⁿ⁻)_{c} or formula (III) (V) (VII), wherein 1>a>0.4 and 0<d<2.

Since phosphate binding will also take place at the surface of the M(II) depleted solid, the amount of surface area is one important attribute in determining how much phosphate the M(II) depleted compound can bind. In embodiments, a surface area can be more than 10 m²/g, or more than 50 m²/g, or more than 100 m²/g, or more than 250 m²/g.

In embodiments, bivalent metal depleted compounds can be made by acid treatment with hydrochloric acid of a suitable starting material as hereinbefore described. Optionally other chemicals may be employed to prepare the substance of disclosure such as using other acids and chelating agents. Optionally other preparation-routes may be used such as treatment of slurries, moist filtration cakes containing the compound, wet-cakes, milled, un-milled forms of the dried compound or even by controlling the pH during the reaction-stage. Preferably, at a pH less than 10 but not less than pH=3; between this range pH 5 is preferred. Optionally, the recipe for the co-precipitation route may be changed by using a smaller amount of the bivalent salt (i.e. MgSO₄). Optionally other conditions may be used for example high or low temperature or pressure conditions.

The starting material may be prepared by heat treatment (calcination) of the starting material. Alternatively, the depleted material may be heat-treated (calcination) preferably at temperatures equal to or less than 500° C. to improve phosphate binding. Calcination temperatures of equal to or less than 500° C. are preferred to avoid formation of spinel type compounds and optimize phosphate binding. It is preferred that the material is washed in order to remove the water-soluble salts that are the by-product of the treatment. If significant amounts of these soluble salts are left admixed with the isolated solid, then the subsequent solid may potentially have an adverse effect on its phosphate binding behavior. The material is preferably washed such that the remaining level of water soluble salts (having a solubility in water of 1 g/liter or more) is less than 15%, or less than 10%, or less than 5% by weight of the solid mixed metal compound after drying as described below. Especially because of the depletion process (for example with acid treatment with HCI) water-soluble salts of bivalent metals (e.g., MgCI₂) are formed which are the by-product of the depletion treatment. In embodiments, a larger number of repeat wash cycles may be required to remove the water-soluble salts.

After isolation of the depleted compound (with any means of isolation such as filtration, centrifugation or decantation) and washing, the drying is preferably carried out at low temperature (such as to provide a product or oven temperature of up to 120° C.), for example by oven drying, spray drying or fluid bed drying.

Optionally, the dry material may be classified prior to acid-treatment, to remove oversize particles by milling and/or sieving and/or any other suitable technique. In embodiments, for example, the dry material may be processed to restrict the material to be treated to particles which are substantially no greater than 100 μm in diameter. In embodiments, as measured by sieving, less than 10% by weight of particles are greater than 106 μm in diameter, more preferably less than 5%. In embodiments, no particles are greater than 106 μm in diameter as measured by sieving.

The dry material can be directly subjected to the necessary treatment, e.g. with HCI of concentration 0.01 M to 5 M, 0.1 to 2 M, or 0.5 to 1.5 M, for a period of 5 minutes or longer, 15 minutes or longer, or 1 hour or longer. In embodiments, the compound is treated for 1 hour or less, or 30 minutes or less, or 15 minutes or less.

Optionally, the moist filter cake or slurry material may be directly subjected to the treatment. An example process of preparing a bivalent metal depleted compound is provided below:

Taking (20 g of) compound comprising a compound of formula (II) M^{II}₁₋ₐM^{III}ₐO_{b}Aⁿ⁻_{c}.zH₂O (II), where the value of a is from 0.2 to 0.4; or formula (I): M^{II}₁₋ₐM^{III}ₐ(OH)^{2An-}_{c·zHO} (l) where 0<a<0.4 and slurrying in water (500 ml), maintaining the material at a constant maintained pH value selected from the range between 3 to 9, between 4 to 8, or between 5 to 7 for 60 mins, 30 mins, or 15 mins or less with an acid and/or chelating agent, for example HCI, at a concentration of 0.01 M to 5 M, 0.1 to 2 M, or 0.5 to 1.5 M. For example, the acid and/or chelating agent can be 1 M HCI. The slurry is then filtered and washed with (200 ml) of water, or 200 ml or more, or 600 ml or more, or 3000 ml or more. After the filtering or centrifuging and washing, the drying is preferably carried out at low temperature (such as providing a product temperature of up to 120° C.), for example by oven drying, spray drying or fluid bed drying. Oversize particles are then size reduced by milling and/or removed by sieving and/or any other suitable technique, for example to restrict the material to particles which are substantially no greater than 100 μm in diameter. In embodiments, as measured by sieving, the materials has less than 10% by weight of particles that are greater than 106 μm in diameter, or less than 5%, or no particles that are greater than 106 μm in diameter.

In embodiments, the treatment results in a reduction in the amount of loss into solution of metal M^{II} from the acid-treated compound by any desired amount, e.g. at least 1% by weight compared to loss from the untreated compound, when measuring the loss of metal M^{II} using the test as hereinafter described, or at least 2% by weight, or at least 3% by weight, or at least 4% by weight, or at least 5% by weight.

The process mentioned above for making the starting material or making the bivalent metal depleted compounds may also cause other materials to be present in the intermediate product and/or in the final product, for example single (as opposed to mixed) metal compounds which may also be formed during the co-precipitation or depletion process.

### Formulations of Mixed Metal Compounds

Any of the mixed metal compounds described herein can be compounded with one or more additional ingredients or pharmaceutical excipients to make compositions, e.g. granules, tablets, and liquid formulations. In embodiments, a final unit dose can include granules of the mixed metal compound and any other material making up the final unit dose. In embodiments, as a whole, the final unit dose can be free from aluminum and/or free from calcium, using the definitions as detailed above.

As mentioned above, the solid mixed metal compound or compounds may be suitably made by co-precipitation from a solution, e.g. as described in WO 99/15189, followed by centrifugation or filtration, then drying, milling and sieving. The mixed metal compound can then be rewetted again as part of a formulation process to make a composition, e.g. a wet-granulation process, and the resulting granules dried in a fluid-bed. The degree of drying in the fluid-bed is used to establish the desired water content of the formulation, e.g. a tablet.

Mixed metal compounds and formulations containing the same can be used preparation of a medicament for a method or use described herein. The compounds can be formulated in any suitable pharmaceutical composition form but especially in a form suitable for oral administration for example in solid unit dose form such as tablets, capsules, or in liquid form such as liquid (optionally aqueous) suspensions, including the liquid formulation described herein below. However, dosage forms adapted for extra-corporeal or even intravenous administration are also possible. Suitable formulations can be produced by known methods using conventional solid carriers such as, for example, lactose, starch or talcum or liquid carriers such as, for example, water, fatty oils or liquid paraffins. Other carriers which may be used include materials derived from animal or vegetable proteins, such as the gelatins, dextrins and soy, wheat and psyllium seed proteins; gums such as acacia, guar, agar, and xanthan; polysaccharides; alginates; carboxymethylcelluloses; carrageenans; dextrans; pectins; synthetic polymers such as polyvinylpyrrolidone; polypeptide/protein or polysaccharide complexes such as gelatin-acacia complexes; sugars such as mannitol, dextrose, galactose and trehalose; cyclic sugars such as cyclodextrin; inorganic salts such as sodium phosphate, sodium chloride and aluminum silicates; and amino acids having from 2 to 12 carbon atoms such as a glycine, L-alanine, L-aspartic acid, L-glutamic acid, L-hydroxyproline, L-isoleucine, L-leucine and L-phenylalanine. In embodiments, a substance or medicament can include greater than 30%, greater than 50% by weight of a compound or compounds of formula (I) and/or formula (II), e.g. up to 95% or 90% by weight of the substance.

In embodiments, the mixed metal compound can be provided in a unit dose with a one or more pharmaceutically acceptable carriers. A pharmaceutically acceptable carrier may be any material with which the mixed metal compound is formulated to facilitate its administration. A carrier may be a solid or a liquid, including a material which is normally gaseous but which has been compressed to form a liquid, and any of the carriers normally used in formulating pharmaceutical compositions may be used. In embodiments, compositions can contain 0.5% to 95% by weight of active ingredient. The term pharmaceutically acceptable carrier encompasses diluents, excipients or adjuvants.

When the mixed metal compounds are part of a pharmaceutical composition, they can be formulated in any suitable pharmaceutical composition form e.g. powders, granules, granulates, sachets, capsules, stick packs, battles, tablets but especially in a form suitable for oral administration for example in solid unit dose form such as tablets, capsules, or in liquid form such as liquid suspensions, especially aqueous suspensions or semi-solid formulations, e.g. gels, chewy bar, dispersing dosage, chewable dosage form or edible sachet. Direct addition to food may also be possible.

Dosage forms adapted for extra-corporeal or even intravenous administration are also possible. Suitable formulations can be produced by known methods using conventional solid carriers such as, for example, lactose, starch or talcum or liquid carriers such as, for example, water, fatty oils or liquid paraffins. Other carriers which may be used include materials derived from animal or vegetable proteins, such as the gelatins, dextrins and soy, wheat and psyllium seed proteins; gums such as acacia, guar, agar, and xanthan; polysaccharides; alginates; carboxymethylcelluloses; carrageenans; dextrans; pectins; synthetic polymers such as polyvinylpyrrolidone; polypeptide/protein or polysaccharide complexes such as gelatin-acacia complexes; sugars such as mannitol, dextrose, galactose and trehalose; cyclic sugars such as cyclodextrin; inorganic salts such as sodium phosphate, sodium chloride and aluminum silicates; and amino acids having from 2 to 12 carbon atoms such as a glycine, L-alanine, L-aspartic acid, L-glutamic acid, L-hydroxyproline, L-isoleucine, L-leucine and L-phenylalanine.

Auxiliary components such as tablet disintegrants, solubilizes, preservatives, antioxidants, surfactants, viscosity enhancers, coloring agents, flavoring agents, pH modifiers, sweeteners or taste-masking agents may also be incorporated into the composition. Suitable coloring agents include red, black and yellow iron oxides and FD & C dyes such as FD & C blue No. 2 and FD & C red No. 40 available from Ellis & Everard. Suitable flavoring agents include mint, raspberry, liquorice, orange, lemon, grapefruit, caramel, vanilla, cherry and grape flavors and combinations of these. Suitable pH modifiers include sodium hydrogencarbonate, citric acid, tartaric acid, hydrochloric acid and maleic acid. Suitable sweeteners include aspartame, acesulfame K and thaumatin. Suitable taste-masking agents include sodium hydrogencarbonate, ion-exchange resins, cyclodextrin inclusion compounds, adsorbates or microencapsulated actives.

In embodiments, a mixed metal compound may be used as the sole active ingredient or in combination with another active ingredient. For example, a mixed metal compound may be used in combination with a vitamin D, e.g. a 25-hydroxyvitamin D compound, e.g. 25-hydroxyvitamin D₃ in immediate or controlled (e.g. sustained or extended) release form.

As described in detail below, any of the compound disclosed herein can be prepared in the form of granulates. In embodiments, when comprised in the granulate form, 90% of the compound based on volume (d90) can have a particle size of less than 1000 micron, for example, 90% of the compound based on volume (d90) can have a particle size of less than 750 micron, for example, 90% of the compound based on volume (d90) can have a particle size of less than 500 micron, for example, 90% of the compound based on volume (d90) can have a particle size of less than 250 micron.

The water content of the granules of is expressed in terms of the content of non-chemically bound water in the granules. This non-chemically bound water therefore excludes chemically bound water. Chemically bound water may also be referred to as structural water.

The amount of non-chemically bound water is determined by pulverizing the granules, heating at 105° C. for 4 hours and immediately measuring the weight loss. The weight equivalent of non-chemically bound water driven off can then be calculated as a weight percentage of the granules.

If the amount of non-chemically bound water is less than 3% by weight of the granules, tablets formed from the granules become brittle and may break very easily. If the amount of non-chemically bound water is greater than 10% by weight of the granules, disintegration time of the granules and of tablets prepared from the granules increases, with an associated reduction in phosphate binding rate and the storage stability of the tablet or granules becomes unacceptable leading to crumbling on storage. The water provided by zH₂O in formula (I) may provide part of the 3 to 12% by weight of non-chemically bound water (based on the weight of the granular material). One skilled in the art may readily determine the value of z based on standard chemical techniques. Once the material has been provided the amount of the non-chemically bound water may then also be readily determined in accordance with the procedure described herein.

The granules can comprise at least 50%, or at least 60%, or at least 70% or at least 75%, by weight inorganic phosphate binder.

The granules can comprise from 3 to 12% by weight of non-chemically bound water, or from 5 to 10% by weight.

The remainder of the granules comprises a pharmaceutically acceptable carrier for the phosphate binder, chiefly an excipient or blend of excipients, which provides the balance of the granules. Hence, the granules may comprise no greater than 47% by weight of excipient. For example, the granules can comprise from 5 to 47% by weight of excipient, or from 10 to 47% by weight of excipient, or from 15 to 47% by weight of excipient.

Suitably, at least 95% by weight of the granules have a diameter less than 1180 micrometers as measured by sieving. Optionally, at least 50% by weight of the granules have a diameter less than 710 micrometers as measured by sieving. Further, optionally, at least 50% by weight of the granules have a diameter from 106 to 1180 micrometers, or from 106 to 500 micrometers. Further, optionally, at least 70% by weight of the granules have a diameter from 106 to 1180 micrometers, or from 106 to 500 micrometers.

The weight median particle diameter of the granules can be in a range of 200 to 400 micrometers.

Larger granules can lead to unacceptably slow phosphate binding. Too high a proportion of granules less than 106 micrometers in diameter can lead to the problem of poor flowability of the granules. Thus, it is contemplated that at least 50% by weight of the granules can have a diameter greater than 106 micrometers as measured by sieving, or at least 80% by weight.

Examples of excipients which may be included in the granules include conventional solid diluents such as, for example, lactose, starch or talcum, as well as materials derived from animal or vegetable proteins, such as the gelatins, dextrins and soy, wheat and psyllium seed proteins; gums such as acacia, guar, agar, and xanthan; polysaccharides; alginates; carboxymethylcelluloses; carrageenans; dextrans; pectins; synthetic polymers such as polyvinylpyrrolidone; polypeptide/protein or polysaccharide complexes such as gelatin-acacia complexes; sugars such as mannitol, dextrose, galactose and trehalose; cyclic sugars such as cyclodextrin; inorganic salts such as sodium phosphate, sodium chloride and aluminum silicates; and amino acids having from 2 to 12 carbon atoms such as a glycine, L-alanine, L-aspartic acid, L-glutamic acid, L-hydroxyproline, L-isoleucine, L-leucine and L-phenylalanine.

The term excipient herein also includes auxiliary components such as tablet structurants or adhesives, disintegrants or swelling agents.

Examples of structurants for tablets include acacia, alginic acid, carboxymethylcellulose, hydroxyethylcellulose, hydroxypropylcellulose, dextrin, ethylcellulose, gelatin, glucose, guar gum, hydroxypropylmethylcellulose, kaltodectrin, methylcellulose, polyethylene oxide, povidone, sodium alginate and hydrogenated vegetable oils.

Examples of disintegrants include cross-linked disintegrants. For example, suitable disintegrants include cross-linked sodium carboxymethylcellulose, cross-linked hydroxypropylcellulose, high molecular weight hydroxypropylcellulose, carboxymethylamide, potassium methacrylatedivinylbenzene copolymer, polymethylmethacrylate, cross-linked polyvinylpyrrolidone (PVP) and high molecular weight polyvinylalcohols.

In embodiments, the granule can include cross-linked polyvinylpyrrolidone (also known as crospovidone, for example available as Kollidon CL-M™ ex BASF). In embodiments, the granules comprise from 1 to 15% by weight of cross-linked polyvinylpyrrolidone 1 to 10%, 2 to 8%. The cross-linked polyvinylpyrrolidone can have a d50 weight median particle size, prior to granulation of less than 50 micrometers (i.e. so-called B-type cross-linked PVP). Such material is also known as micronised crospovidone. The cross-linked polyvinylpyrrolidone at these levels leads to good disintegration of the tablet but with less inhibition of phosphate binding of the inorganic phosphate binder as compared to some other excipients. The micronized sizes for the cross-linked polyvinylpyrollidone give reduced grittiness and hardness of the particles formed as the tablets disintegrate.

In embodiments, the granule can include pregelatinised starch (also known as pregelled starch). In embodiments, the granules comprise from 5 to 20% by weight of pregelled starch, 10 to 20%, from 12 to 18% by weight. The pregelatinised starch at these levels can improve the durability and cohesion of the tablets without impeding the disintegration or phosphate binding of the tablets in use. The pregelatinised starch can be fully pregelatinised, with a moisture content from 1 to 15% by weight and a weight median particle diameter from 100 to 250 micrometers. An example material is Lycotab™—a fully pregelatinised maize starch available from Roquette.

The granules may also comprise preservatives, wetting agents, antioxidants, surfactants, effervescent agents, coloring agents, flavoring agents, pH modifiers, sweeteners or taste-masking agents. Suitable coloring agents include red, black and yellow iron oxides and FD & C dyes such as FD & C blue No. 2 and FD & C red No. 40 available from Ellis & Everard. Suitable flavoring agents include mint, raspberry, liquorice, orange, lemon, grapefruit, caramel, vanilla, cherry and grape flavors and combinations of these. Suitable pH modifiers include sodium hydrogencarbonate (i.e. bicarbonate), citric acid, tartaric acid, hydrochloric acid and maleic acid. Suitable sweeteners include aspartame, acesulfame K and thaumatin. Suitable taste-masking agents include sodium hydrogencarbonate, ion-exchange resins, cyclodextrin inclusion compounds and adsorbates. Suitable wetting agents include sodium lauryl sulphate and sodium docusate. A suitable effervescent agent or gas producer is a mixture of sodium bicarbonate and citric acid.

Granulation may be performed by a process comprising the steps of:

i) mixing the solid water-insoluble inorganic compound capable of binding phosphate with one or more excipients to produce a homogeneous mix,

ii) contacting a suitable liquid with the homogeneous mix and mixing in a granulator to form wet granules,

iii) optionally passing the wet granules though a screen to remove granules larger than the screen size,

iv) drying the wet granules to provide dry granules.

v) milling and/or sieving the dry granules.

Suitably the granulation is by wet granulation, comprising the steps of;

i) mixing the inorganic solid phosphate binder with suitable excipients to produce a homogeneous mix,

ii) adding a suitable liquid to the homogeneous mix and mixing in a granulator to form granules,

iii) optionally passing the wet granules though a screen to remove granules larger than the screen size,

iv) drying the granules.

v) milling and sieving the granules

Suitable liquids for granulation include water, ethanol and mixtures thereof. Water is a preferred granulation liquid.

The granules are dried to the desired moisture levels as described hereinbefore prior to their use in tablet formation or incorporation into a capsule for use as a unit dose.

A solid unit dose form may also comprise a release rate controlling additive. For example, the mixed metal compound may be held within a hydrophobic polymer matrix so that it is gradually leached out of the matrix upon contact with body fluids. Alternatively, the mixed metal compound may be held within a hydrophilic matrix which gradually or rapidly dissolves in the presence of body fluid. The tablet may comprise two or more layers having different release properties. The layers may be hydrophilic, hydrophobic or a mixture of hydrophilic and hydrophobic layers. Adjacent layers in a multilayer tablet may be separated by an insoluble barrier layer or hydrophilic separation layer. An insoluble barrier layer may be formed of materials used to form the insoluble casing. A hydrophilic separation layer may be formed from a material more soluble than the other layers of the tablet core so that as the separation layer dissolves the release layers of the tablet core are exposed.

Suitable release rate controlling polymers include polymethacrylates, ethylcellulose, hydroxypropylmethylcellulose, methylcellulose, hydroxyethylcellulose, hydroxypropylcellulose, sodium carboxymethylcellulose, calcium carboxymethylcellulose, acrylic acid polymer, polyethylene glycol, polyethylene oxide, carrageenan, cellulose acetate, zein etc.

Suitable materials which swell on contact with aqueous liquids include polymeric materials include from cross-linked sodium carboxymethylcellulose, cross-linked hydroxypropylcellulose, high molecular weight hydroxypropylcellulose, carboxymethylamide, potassium methacrylatedivinylbenzene copolymer, polymethylmethacrylate, cross-linked polyvinylpyrrolidone and high molecular weight polyvinylalcohols. Solid unit dose forms comprising a mixed metal compound may be packaged together in a container or presented in foil strips, blister packs or the like, e.g. marked with days of the week against respective doses, for patient guidance.

Solid unit dose forms comprising a mixed metal compound may be packaged together in a container or presented in foil strips, blister packs or the like, e.g. marked with days of the week against respective doses, for patient guidance.

There is also a need for formulations which could improve patient compliance, for example in case of elderly or pediatric patients. A formulation in powder dose form could be either diluted in water, reconstituted or dispersed.

A process for the preparation of a pharmaceutical composition as described herein is also provided, which comprises bringing at least one mixed metal compound into association with a pharmaceutically acceptable carrier and optionally, any other ingredients including by-products resulting from manufacture of the active ingredient.

A pharmaceutically acceptable carrier may be any material with which the mixed metal compound is formulated to facilitate its administration. A carrier may be a solid or a liquid, including a material which is normally gaseous but which has been compressed to form a liquid, and any of the carriers normally used in formulating pharmaceutical compositions may be used. In embodiments, compositions can contain 0.5% to 95% by weight of active ingredient. The term pharmaceutically acceptable carrier encompasses diluents, excipients or adjuvants.

Prior to tableting the granules into a unit dose composition, the granules can be blended with a lubricant or glidant such that there is lubricant or glidant distributed over and between the granules during the compaction of the granules to form tablets.

The optimum amount of lubricant required can depend on the lubricant particle size and on the available surface area of the granules. Suitable lubricants include silica, talc, stearic acid, calcium or magnesium stearate and sodium stearyl fumarate and mixtures thereof. Lubricants are added to the granules in a finely divided form, typically no particles greater than 40 micrometers in diameter (ensured typically by sieving). The lubricant is suitably added to the granules at a level of from 0.1 to 0.4%, or from 0.2 to 0.3% by weight of the granules. Lower levels can lead to sticking or jamming of the tablet die whereas higher levels may reduce the rate of phosphate binding or hinder tablet disintegration. Salts of fatty acids may be used as lubricants, such as calcium and/or magnesium stearate. A lubricant can be selected from the group consisting of magnesium stearate, sodium stearyl fumarate and mixtures thereof. Some lubricants, such as fatty acids, lead to pitting and loss of integrity in the coating layer of the tablets. It is thought that this may arise from partial melting of the lubricant as the coating layer is dried. Hence, in some embodiments the lubricant has a melting point in excess of 55° C.

In embodiments, tablets may be prepared by compressing granules, under high pressure, in order to form a tablet having the necessary crushing strength for the handling required during packaging and distribution. The use of granules formed from a granulated powder mixture improves flowability from storage hoppers to the tableting press, which in turn benefits the efficiency of tablet processing. The inorganic phosphate binders used in the tablets can typically have poor flowability properties at their desired particle size as detailed hereinbefore. Because it is desired that the tablets have high levels of inorganic phosphate binder, of the order of 50% or more by weight of the tablet, the inorganic phosphate binder cab be formed into granules prior to tablet formation. A fine powder is apt to pack or “bridge” in the hopper, feed shoe or die, and thus tablets of even weight or even compression are not easily obtainable. Even if it were possible to compress fine powders to a satisfactory degree, air may be trapped and compressed, which may lead to splitting of the tablet on ejection. The use of granules helps to overcome these problems. Another benefit of granulation is the increase in bulk density of the final tablet when prepared from granules rather than from fine powder, reducing the size of the final tablet and improving the likelihood of patient compliance.

In embodiments, tablets may be circular or can be generally bolus- or torpedo-shaped (also known as double convex oblong shaped tablet,) i.e. having an elongate dimension, in order to assist swallowing of larger doses. It may for example be in the form of a cylinder with rounded ends or elliptical in one dimension and circular in an orthogonal dimension, or elliptical in both. Some flattening of one or more parts of the overall shape is also possible.

Where the tablet is in the form of a tablet provided with a “belly-band”, it is contemplated that the width of the belly-band is 2 mm or more. Smaller belly-bands can lead to insufficient coverage or chipping or loss of integrity of the water-resistant coating of the tablet.

In embodiments, tablets can have a hardness from 5 to 30 kgf as measured using a Holland C50 tablet hardness tester.

In embodiments, tablets, once formed can be provided with a water-resistant coating.

The water-resistant coating may be applied to the tablet by any of the usual pharmaceutical coating processes and equipment. For example, tablets may be coated by fluid bed equipment (for example a “Wurster” type fluid bed dryer) coating pans (rotating, side vented, convention etc), with spray nozzles or guns or other sprayer types or by dipping and more recent techniques including Supercell tablet coater from Niro PharmaSystems. Variations in available equipment include size, shape, location of nozzles and air inlets and outlets, air flow patterns and degree of instrumentation. Heated air may be used to dry the sprayed tablets in a way that allows continuous spraying while the tablets are being simultaneously dried. Discontinuous or intermittent spraying may also be used, but generally requires longer coating cycles. The number and position of nozzles may be varied, as needed depending on the coating operation and the nozzles(s) is preferably aimed perpendicularly or nearly perpendicular to the bed although other direction(s) of aim may be employed if desired. A pan may be rotated at a speed selected from a plurality of operating speeds. Any suitable system capable of applying a coating composition to a tablet may be used. Virtually any tablet is acceptable herein as a tablet to be coated. The term “tablet” could include tablet, pellet or pill. The tablet can be in a form sufficiently stable physically and chemically to be effectively coated in a system which involves some movement of a tablet, as for example in a fluidized bed, such as in a fluidized bed dryer or a side vented coating pan, combinations thereof and the like. Tablets may be coated directly, i.e. without a subcoat to prepare the surface. Subcoats or topcoats may of course be used. If desired, the same or a similar coating application system can be employed for both a first or second or more coating applications. The coating composition is prepared according to the physical properties of its constituents, i.e. soluble materials are dissolved, insoluble materials are dispersed. The type of mixing used is also based on the properties of the ingredients. Low shear liquid mixing is used for soluble materials and high shear liquid mixing is used for insoluble materials. Usually the coating formulation consists of two parts, the colloidal polymer suspension and the pigment suspension or solution (e.g. red oxide or Quinoline yellow dye). These are prepared separately and mixed before use.

A wide range of coating materials may be used, for example, cellulose derivatives, polyvinylpyrrolidone, polyvinyl alcohol, polyvinyl acetate, polyethylene glycols, copolymers of styrene and acrylate, copolymers of acrylic acid and methacrylic acid, copolymers of methacrylic acid and ethylacrylate, copolymers of methyl methacrylate and methacrylate, copolymers of methacrylate and tertiary amino alkyl methacrylate, copolymers of ethylacrylate methyl methacrylate and quaternary amino alkyl methacrylate and combinations of two or more hereof. Salts of methacrylate copolymers can be used, e.g. butylated methacrylate copolymer (commercially available as Eudragit EPO).

The coating is suitably present as 0.05 to 10% by weight of the coated tablet, or from 0.5% to 7%. The coating material can be used in combination with red iron oxide pigment (Fe²O³) (1% or more, or 2% or more by weight of the dried coating layer) which is dispersed throughout the coating material and provides an even coloring of the coating layer on the tablet giving a pleasant uniform appearance.

In addition to protecting the tablet core from moisture loss or ingress on storage, the water resistant coating layer also helps to prevent the rapid breakup of the tablet in the mouth, delaying this until the tablet reaches the stomach. With this purpose in mind, it is preferred if the coating material has low solubility in alkaline solution such as found in the mouth, but more soluble in neutral or acid solution. Contemplated coating polymers include salts of methacrylate copolymers, particularly butylated methacrylate copolymer (commercially available as Eudragit EPO). The coating layer can comprise at least 30% by weight of a coating polymer, or at least 40% by weight.

The water loss or uptake of coated tablets is suitably measured as detailed hereinbefore for the measurement of the non-chemically bound water content for granules. From a set of freshly prepared coated tablets, some are measured for non-chemically bound water immediately following preparation, and others are measured after storage as detailed above.

In embodiments, a tablet can be made by granulating a water-insoluble inorganic solid phosphate binder with a pharmaceutically acceptable excipient and optionally, any other ingredients, forming a tablet from the granules by compression and optionally applying a water-resistant coating to the tablet so formed.

In embodiments, the pharmaceutical composition, such as granules, can be provided in capsules. For example, a hard gelatin capsules can be used. Other suitable capsule films can be used as well.

A tablet for human adult administration can comprise from 1 mg to 5 g, or from 10 mg to 2 g, or from 100 mg to 1 g, such as from 150 mg to 750 mg, from 200 mg to 750 mg or from 250 mg to 750 mg of water-insoluble inorganic solid mixed metal compound, for example.

In embodiments, unit doses can include at least 200 mg of a water-insoluble solid inorganic mixed metal compound. In embodiments, unit doses can include at least 250 mg, at least 300 mg, at least 500 mg, at least 700 mg, at least 750 mg of a water-insoluble solid inorganic mixed metal compound. In embodiments, the unit dose can contain 200 mg (±20 mg), 250 mg (±20 mg), or 300 mg (±20 mg) of a water-insoluble solid inorganic mixed metal compound. When the unit dose is a tablet, the unit dose weight includes any optional coating.

The tablet forms may be packaged together in a container or presented in foil strips, blister packs or the like, e.g. marked with days of the week against respective doses, for patient guidance.

Any of disclosed the mixed metal compounds can be for use in or as a medicine on humans or animals. Any of disclosed the mixed metal compounds can be used in the manufacture of a medicament for use on animals or humans in the treatment or therapy of a condition or disease as described herein.

As discussed herein, mixed metal compounds and formulations thereof can be provided in tablets which are stable of over a period of at least 12 months determined at 25° C./60 RH and 30° C./65 RH. Under more extreme storage conditions (40° C./75 RH) the storage stability can be at least 6 months.

The mixed metal compound can also be used in the form of composition which is a liquid formulation. A mixed metal compound for use herein can also be used in the form of a liquid formulation containing water-insoluble inorganic mixed metal compounds. The liquid dosage forms can provide a useful means of administration for subjects who have difficulty swallowing. In particular in the field of pharmaceuticals ease of administration may also help ensure optimal patient compliance. Additionally liquid form allows for a continuously variable dose quantity to be administered.

In a first aspect the liquid formulation comprises:
(i) a water-insoluble mixed metal compound as described herein,
(ii) xanthan gum; and
(iii) at least one of
   (a) polyvinyl pyrrolidone;
   (b) locust bean gum; and
   (c) methyl cellulose
wherein the liquid formulation has been irradiated with ionising radiation in an amount of at least 4 kGy.

The liquid formulation provides a carrier system for delivering insoluble mixed metal compounds, e.g. those containing at least one trivalent metal selected from iron (III) and aluminium and at least one divalent metal selected from of magnesium, iron, zinc, calcium, lanthanum and cerium.

The liquid formulation optionally provides a system in which the use of oil-based carriers is avoided. Such carriers can have the drawback of a high relative calorific value. Such high calorific values are generally considered to be undesirable and are particularly unsuitable for subjects on a calorie restricted diet and/or who may consume the liquid formulation for a prolonged period of time.

The liquid formulation is further advantageous in that it allows for high loads of mixed metal compound to be delivered. This is advantageous in that the volume of product required to deliver a determined amount of mixed metal compound is kept within acceptable amounts. The use of such high loads is particularly advantageous for subjects who desire or are required to control fluid intake. Such a group is patients on dialysis who must typically restrict the volume of liquid which they consume. Any aqueous liquid dose formulation will contribute to the volume of liquid which the patient consumes, hence the volume of liquid must be kept to a minimum.

The liquid formulation is further advantageous in that it provides for a preserved liquid composition wherein the addition of preservative components is not required. By selection of a specific combination of suspension materials and selection of a specific radiation dosage, a stable and preserved liquid formulation may be provided. Mixed metal compounds in an un-buffered aqueous system at a concentration range of interest (e.g., around 10% w/v) provide a relatively high pH (ca. 9.2 to 9.4). The high pH excludes the use of known, commercially available preservatives at concentrations effective for microbial control and at levels that are safe for use in a composition in a human population. For chemical preservation, the pH of the formulation must be limited to about 8.2 or below in order to permit the use of preservatives at concentrations that are safe in the human population. The preservative may have some efficacy above pH 8.2 however there is little margin for pH increase of the formulation, for example, on storage. A significant reduction in pH i.e. below approximately pH 8.0 cannot be made without releasing magnesium from the mixed metal compound structure. This has the effect of changing the mixed metal compound structure and may also impair properties such as phosphate binding performance of the mixed metal compound.

The mixed metal compound utilised in the liquid formulation may be any mixed metal compound described herein, e.g. one containing at least one trivalent metal selected from iron (III) and aluminium and at least one divalent metal selected from of magnesium, iron, zinc, calcium, lanthanum and cerium. For example, the mixed metal compound can contains at least iron (III) and at least magnesium. Optionally, the mixed metal compound can be free of or substantially free of calcium.

The physical stability of the liquid formulation may be improved by reducing the particle size of the mixed metal compound by e.g. micronisation or wet milling, e.g. to a d50 average particle size of less than 10 μm, or in a range of about 2-10 μm, or in a range of about 2-7 μm, or 5 μm.

The physical stability of the liquid formulation may also be further improved by drying the mixed metal compound prior to incorporation in the liquid formulation.

In one aspect the mixed metal compound is present in the liquid formulation in an amount of 8 to 12 w/v, for example about 10 w/v.

The mixed metal compound may have a particle density (as measured in accordance with method 20) of greater than 1.6 g/ml, or greater than 1.9 g/ml. Moreover, the difference between the particle density of the mixed metal compound and the fluid of the liquid formulation (typically comprised of component (ii) and component (iii)) can be greater than 0.2 g/ml.

As described herein the liquid formulation is irradiated with ionising radiation in an amount of at least 4 kGy. The liquid formulation can be irradiated with ionising radiation in an amount of at least 6 kGy, such as in an amount of at least 8 kGy, or such as in an amount of at least 10 kGy. Optionally, the liquid formulation can be irradiated with ionising radiation in an amount of no greater than 20 kGy, such as in an amount of no greater than 15 kGy, such as in an amount of no greater than 12 kGy, or in an amount of no greater than 10 kGy. The liquid formulation may be irradiated with ionising radiation in an amount of 1 to 15 kGy, such as 2 to 14 kGy, such as 4 to 12 kGy, or 6 to 10 kGy. In other aspects, liquid formulation can be one that has been irradiated with ionising radiation in an amount of from 4 to 20 kGy, such as in an amount of from 4 to 15 kGy, such as in an amount of from 4 to 12 kGy, or in an amount of from 4 to 10 kGy. Optionally the liquid formulation has been irradiated with ionising radiation in an amount of from 6 to 20 kGy, such as in an amount of from 6 to 15 kGy, such as in an amount of from 6 to 12 kGy, or in an amount of from 6 to 10 kGy.

Any suitable source of ionising irradiation may be used to provide the desired level of irradiation. It is contemplated that electron beam, gamma and x-ray irradiation will be suitable.

Xanthan gum is a natural anionic biopolysaccharide made up of different monosacharides, mannose, glucose and glucuronic acids. It has the advantage over other common natural polymers of resisting degradation by enzymes. Suspensions using xanthan gums have the advantage that once the yield stress is exceeded, they are shearing thinning i.e. the viscosity reduces with increasing shear input. Therefore, if settling occurs, shear input can be applied (by, for example shaking of the liquid container) to reduce the viscosity and thus aid re-dispersion of any settled solids. As discussed herein, the present liquid formulation contains xanthan gum. One skilled in the art will appreciate that the xanthan gum may be present in any suitable amount sufficient to achieve one or more goals described herein.

In one aspect the xanthan gum is present in an amount of no greater than 10 wt %, or in an amount of no greater than 7 wt %, or in an amount of no greater than 5 wt %, or in an amount of no greater than 3 wt %, or in an amount of no greater than 2 wt %, or in an amount of no greater than 1.5 wt %, or in an amount of no greater than lwt %, or in an amount of no greater than 0.8 wt %, or in an amount of no greater than 0.6 wt %, or in an amount of no greater than 0.5 wt % based on weight of the liquid formulation.

In one aspect the xanthan gum is present in an amount of no less than 0.01 wt %, or in an amount of no less than 0.02 wt %, or in an amount of no less than 0.03 wt %, or in an amount of no less than 0.05 wt %, or in an amount of no less than 0.08 wt %, or in an amount of no less than 0.1 wt %, or in an amount of no less than 0.2 wt %, or in an amount of no less than 0.3 wt % based on weight of the liquid formulation.

In one aspect the xanthan gum is present in an amount of from 0.01 to 10 wt %, or in an amount of from 0.02 to 7 wt %, or in an amount of from 0.03 to 5 wt %, or in an amount of from 0.05 to 3 wt %, or in an amount of from 0.08 to 2 wt %, or in an amount of from 0.1 to 1 wt %, or in an amount of from 0.2 to 0.8 wt %, or in an amount of from 0.2 to 0.6 wt %, or in an amount of from 0.2 to 0.5 wt %, or in an amount of from 0.3 to 0.5 wt % based on weight of the liquid formulation.

As discussed herein, the liquid formulation contains at least one of (a) polyvinyl pyrrolidone, (b) locust bean gum, and (c) methyl cellulose. It will be appreciated by one skilled in the art that by at least of it is meant that one of the listed components may be present, two of the listed components may be present or all three of the listed components may be present. The one, two or three listed components may be present in any suitable amount sufficient to achieve one or more goals described herein.

In one aspect the liquid formulation contains polyvinyl pyrrolidone. In one aspect the liquid formulation contains locust bean gum. In one aspect the liquid formulation contains methyl cellulose. In one aspect the liquid formulation contains polyvinyl pyrrolidone and locust bean gum. In one aspect the liquid formulation contains polyvinyl pyrrolidone and methyl cellulose. In one aspect the liquid formulation contains locust bean gum and methyl cellulose.

In one aspect the liquid formulation contains polyvinyl pyrrolidone, locust bean gum, and methyl cellulose.

Locust bean gum is a high molecular weight, hydrophilic polysaccharide. It is non-ionic and is therefore unlikely to compete with phosphate by binding to the mixed metal compound.

In one aspect component (iii) is present in an amount of no greater than 10 wt %, or in an amount of no greater than 7 wt %, or in an amount of no greater than 5 wt %, or in an amount of no greater than 3 wt %, or in an amount of no greater than 2 wt %, or in an amount of no greater than 1.5 wt %, or in an amount of no greater than lwt %, or in an amount of no greater than 0.8 wt %, or in an amount of no greater than 0.6 wt %, or in an amount of no greater than 0.5 wt % based on weight of the liquid formulation. It will be understood that each of the above amounts refers to the combined total amount of (a) polyvinyl pyrrolidone, (b) locust bean gum, and (c) methyl cellulose.

In one polyvinyl pyrrolidone is present in an amount of no greater than 10 wt %, or in an amount of no greater than 7 wt %, or in an amount of no greater than 5 wt %, or in an amount of no greater than 3 wt %, or in an amount of no greater than 2 wt %, or in an amount of no greater than 1.5 wt %, or in an amount of no greater than lwt %, or in an amount of no greater than 0.8 wt %, or in an amount of no greater than 0.6 wt %, or in an amount of no greater than 0.5 wt % based on weight of the liquid formulation.

In one aspect locust bean gum is present in an amount of no greater than 10 wt %, or in an amount of no greater than 7 wt %, or in an amount of no greater than 5 wt %, or in an amount of no greater than 3 wt %, or in an amount of no greater than 2 wt %, or in an amount of no greater than 1.5 wt %, or in an amount of no greater than 1 wt %, or in an amount of no greater than 0.8 wt %, or in an amount of no greater than 0.6 wt %, or in an amount of no greater than 0.5 wt % based on weight of the liquid formulation.

In one aspect methyl cellulose is present in an amount of no greater than 10 wt %, or in an amount of no greater than 7 wt %, or in an amount of no greater than 5 wt %, or in an amount of no greater than 3 wt %, or in an amount of no greater than 2 wt %, or in an amount of no greater than 1.5 wt %, or in an amount of no greater than 1 wt %, or in an amount of no greater than 0.8 wt %, or in an amount of no greater than 0.6 wt %, or in an amount of no greater than 0.5 wt % based on weight of the liquid formulation.

In one aspect component (iii) is present in an amount of no less than 0.01 wt %, or in an amount of no less than 0.02 wt %, or in an amount of no less than 0.03 wt %, or in an amount of no less than 0.05 wt %, or in an amount of no less than 0.08 wt %, or in an amount of no less than 0.1 wt %, or in an amount of no less than 0.2 wt %, or in an amount of no less than 0.3 wt % based on weight of the liquid formulation. It will be understood that each of the above amounts refers to the combined total amount of (a) polyvinyl pyrrolidone, (b) locust bean gum, and (c) methyl cellulose.

In one aspect polyvinyl pyrrolidone is present in an amount of no less than 0.01 wt %, or in an amount of no less than 0.02 wt %, or in an amount of no less than 0.03 wt %, or in an amount of no less than 0.05 wt %, or in an amount of no less than 0.08 wt %, or in an amount of no less than 0.1 wt %, or in an amount of no less than 0.2 wt %, or in an amount of no less than 0.3 wt % based on weight of the liquid formulation.

In one locust bean gum is present in an amount of no less than 0.01 wt %, or in an amount of no less than 0.02 wt %, or in an amount of no less than 0.03 wt %, or in an amount of no less than 0.05 wt %, or in an amount of no less than 0.08 wt %, or in an amount of no less than 0.1 wt %, or in an amount of no less than 0.2 wt %, or in an amount of no less than 0.3 wt % based on weight of the liquid formulation.

In one aspect methyl cellulose is present in an amount of no less than 0.01 wt %, or in an amount of no less than 0.02 wt %, or in an amount of no less than 0.03 wt %, or in an amount of no less than 0.05 wt %, or in an amount of no less than 0.08 wt %, or in an amount of no less than 0.1 wt %, or in an amount of no less than 0.2 wt %, or in an amount of no less than 0.3 wt % based on weight of the liquid formulation.

In one aspect component (iii) is present in an amount of from 0.01 to 10 wt %, or in an amount of from 0.02 to 7 wt %, or in an amount of from 0.03 to 5 wt %, or in an amount of from 0.05 to 3 wt %, or in an amount of from 0.08 to 2 wt %, or in an amount of from 0.1 to 1 wt %, or in an amount of from 0.2 to 0.8 wt %, or in an amount of from 0.2 to 0.6 wt %, or in an amount of from 0.2 to 0.5 wt %, or in an amount of from 0.3 to 0.5 wt % based on weight of the liquid formulation. It will be understood that each of the above amounts refers to the combined total amount of (a) polyvinyl pyrrolidone, (b) locust bean gum, and (c) methyl cellulose.

In one aspect polyvinyl pyrrolidone is present in an amount of from 0.01 to 10 wt %, or in an amount of from 0.02 to 7 wt %, or in an amount of from 0.03 to 5 wt %, or in an amount of from 0.05 to 3 wt %, or in an amount of from 0.08 to 2 wt %, or in an amount of from 0.1 to 1 wt %, or in an amount of from 0.2 to 0.8 wt %, or in an amount of from 0.2 to 0.6 wt %, or in an amount of from 0.2 to 0.5 wt %, or in an amount of from 0.3 to 0.5 wt % based on weight of the liquid formulation.

In one aspect locust bean gum is present in an amount of from 0.01 to 10 wt %, or in an amount of from 0.02 to 7 wt %, or in an amount of from 0.03 to 5 wt %, or in an amount of from 0.05 to 3 wt %, or in an amount of from 0.08 to 2 wt %, or in an amount of from 0.1 to 1 wt %, or in an amount of from 0.2 to 0.8 wt %, or in an amount of from 0.2 to 0.6 wt %, or in an amount of from 0.2 to 0.5 wt %, or in an amount of from 0.3 to 0.5 wt % based on weight of the liquid formulation.

In one aspect methyl cellulose is present in an amount of from 0.01 to 10 wt %, or in an amount of from 0.02 to 7 wt %, or in an amount of from 0.03 to 5 wt %, or in an amount of from 0.05 to 3 wt %, or in an amount of from 0.08 to 2 wt %, or in an amount of from 0.1 to 1 wt %, or in an amount of from 0.2 to 0.8 wt %, or in an amount of from 0.2 to 0.6 wt %, or in an amount of from 0.2 to 0.5 wt %, or in an amount of from 0.3 to 0.5 wt % based on weight of the liquid formulation.

Optionally, the palatability of the liquid formulation may be improved by the addition of one or more sweeteners (either alone or in combination with sorbitol) and/or flavourings. For example, sweeteners such as Acesulfame K/Aspartame, Xylitol, Thaumatin (Talin) and Saccharin; and flavourings such as Butterscotch, Caramel, Vanilla, Mild peppermint and Strawberry, may be used.

The absolute amounts of xanthan gum and component (iii), namely at least one of (a) polyvinyl pyrrolidone (b) locust bean gum and (c) methyl cellulose in the liquid formulation are defined herein in certain optional embodiments. The ratio of xanthan gum and component (iii) may be any suitable ratio within the absolute amounts described herein. In one aspect the xanthan gum and component (iii) are present in a ratio of 2:1 to 1:2. Or the xanthan gum and component (iii) are present in a ratio of approximately 1:1.

When the liquid formulation comprises at least polyvinyl pyrrolidone, the liquid formulation can comprise (ii) xanthan gum and (iii) polyvinyl pyrrolidone, wherein the xanthan gum and polyvinyl pyrrolidone are present in a ratio of approximately 2:1. In this aspect optionally the liquid formulation has been irradiated with ionising radiation in an amount of at least 8 kGy.

When the liquid formulation comprises at least locust bean gum, optionally the liquid formulation comprises (ii) xanthan gum and (iii) locust bean gum, wherein the xanthan gum and locust bean gum are present in a ratio of approximately 1:1. In this aspect the liquid formulation optionally has been irradiated with ionising radiation in an amount of at least 6 kGy.

When the liquid formulation comprises at least methyl cellulose, optionally the liquid formulation comprises (ii) xanthan gum and (iii) methyl cellulose, wherein the xanthan gum and methyl cellulose are present in a ratio of approximately 1:1. In this aspect optionally the liquid formulation has been irradiated with ionising radiation in an amount of at least 10 kGy.

The following liquid formulations are contemplated

Polyvinyl Pyrrolidone Containing Liquid Formulations

| | |
|---|---|
| xanthan gum | polyvinyl pyrrolidone |

based on weight of the liquid formulation

| | |
|---|---|
| from 0.01 to 10 wt % | from 0.01 to 10 wt %; or |
| | from 0.02 to 7 wt %; or |
| | from 0.03 to 5 wt %; or |
| | from 0.05 to 3 wt %; or |
| | from 0.08 to 2 wt %; or |
| | from 0.1 to 1 wt %; or |
| | from 0.2 to 0.8 wt %; or |
| | from 0.2 to 0.6 wt %; or |
| | from 0.2 to 0.5 wt %; or |
| | from 0.3 to 0.5 wt %. |
| from 0.02 to 7 wt % | from 0.01 to 10 wt %; or |
| | from 0.02 to 7 wt %; or |
| | from 0.03 to 5 wt %; or |
| | from 0.05 to 3 wt %; or |
| | from 0.08 to 2 wt %; or |
| | from 0.1 to 1 wt %; or |
| | from 0.2 to 0.8 wt %; or |
| | from 0.2 to 0.6 wt %; or |
| | from 0.2 to 0.5 wt %; or |
| | from 0.3 to 0.5 wt %. |
| from 0.03 to 5 wt % | from 0.01 to 10 wt %; or |
| | from 0.02 to 7 wt %; or |
| | from 0.03 to 5 wt %; or |
| | from 0.05 to 3 wt %; or |
| | from 0.08 to 2 wt %; or |
| | from 0.1 to 1 wt %; or |
| | from 0.2 to 0.8 wt %; or |
| | from 0.2 to 0.6 wt %; or |
| | from 0.2 to 0.5 wt %; or |
| | from 0.3 to 0.5 wt %. |
| from 0.05 to 3 wt % | from 0.01 to 10 wt %; or |
| | from 0.02 to 7 wt %; or |
| | from 0.03 to 5 wt %; or |
| | from 0.05 to 3 wt %; or |
| | from 0.08 to 2 wt %; or |
| | from 0.1 to 1 wt %; or |
| | from 0.2 to 0.8 wt %; or |
| | from 0.2 to 0.6 wt %; or |
| | from 0.2 to 0.5 wt %; or |
| | from 0.3 to 0.5 wt %. |
| from 0.08 to 2 wt % | from 0.01 to 10 wt %; or |
| | from 0.02 to 7 wt %; or |
| | from 0.03 to 5 wt %; or |
| | from 0.05 to 3 wt %; or |
| | from 0.08 to 2 wt %; or |
| | from 0.1 to 1 wt %; or |
| | from 0.2 to 0.8 wt %; or |
| | from 0.2 to 0.6 wt %; or |
| | from 0.2 to 0.5 wt %; or |
| | from 0.3 to 0.5 wt %. |
| from 0.1 to 1 wt % | from 0.01 to 10 wt %; or |
| | from 0.02 to 7 wt %; or |
| | from 0.03 to 5 wt %; or |
| | from 0.05 to 3 wt %; or |
| | from 0.08 to 2 wt %; or |
| | from 0.1 to 1 wt %; or |
| | from 0.2 to 0.8 wt %; or |
| | from 0.2 to 0.6 wt %; or |
| | from 0.2 to 0.5 wt %; or |
| | from 0.3 to 0.5 wt %. |
| from 0.2 to 0.8 wt % | from 0.01 to 10 wt %; or |
| | from 0.02 to 7 wt %; or |
| | from 0.03 to 5 wt %; or |
| | from 0.05 to 3 wt %; or |
| | from 0.08 to 2 wt %; or |
| | from 0.1 to 1 wt %; or |
| | from 0.2 to 0.8 wt %; or |
| | from 0.2 to 0.6 wt %; or |
| | from 0.2 to 0.5 wt %; or |
| | from 0.3 to 0.5 wt %. |
| from 0.2 to 0.6 wt % | from 0.01 to 10 wt %; or |
| | from 0.02 to 7 wt %; or |
| | from 0.03 to 5 wt %; or |
| | from 0.05 to 3 wt %; or |
| | from 0.08 to 2 wt %; or |
| | from 0.1 to 1 wt %; or |
| | from 0.2 to 0.8 wt %; or |
| | from 0.2 to 0.6 wt %; or |
| | from 0.2 to 0.5 wt %; or |
| | from 0.3 to 0.5 wt %. |
| from 0.2 to 0.5 wt % | from 0.01 to 10 wt %; or |
| | from 0.02 to 7 wt %; or |
| | from 0.03 to 5 wt %; or |
| | from 0.05 to 3 wt %; or |
| | from 0.08 to 2 wt %; or |
| | from 0.1 to 1 wt %; or |
| | from 0.2 to 0.8 wt %; or |
| | from 0.2 to 0.6 wt %; or |
| | from 0.2 to 0.5 wt %; or |
| | from 0.3 to 0.5 wt %. |
| from 0.3 to 0.5 wt %. | from 0.01 to 10 wt %; or |
| | from 0.02 to 7 wt %; or |
| | from 0.03 to 5 wt %; or |
| | from 0.05 to 3 wt %; or |
| | from 0.08 to 2 wt %; or |
| | from 0.1 to 1 wt %; or |
| | from 0.2 to 0.8 wt %; or |
| | from 0.2 to 0.6 wt %; or |
| | from 0.2 to 0.5 wt %; or |
| | from 0.3 to 0.5 wt %. |

Locust Bean Gum Containing Liquid Formulations

| | |
|---|---|
| xanthan gum | locust bean gum |

based on weight of the liquid formulation

| | |
|---|---|
| from 0.01 to 10 wt % | from 0.01 to 10 wt %; or |
| | from 0.02 to 7 wt %; or |
| | from 0.03 to 5 wt %; or |
| | from 0.05 to 3 wt %; or |
| | from 0.08 to 2 wt %; or |
| | from 0.1 to 1 wt %; or |
| | from 0.2 to 0.8 wt %; or |
| | from 0.2 to 0.6 wt %; or |
| | from 0.2 to 0.5 wt %; or |
| | from 0.3 to 0.5 wt %. |
| from 0.02 to 7 wt % | from 0.01 to 10 wt %; or |
| | from 0.02 to 7 wt %; or |
| | from 0.03 to 5 wt %; or |
| | from 0.05 to 3 wt %; or |
| | from 0.08 to 2 wt %; or |
| | from 0.1 to 1 wt %; or |
| | from 0.2 to 0.8 wt %; or |
| | from 0.2 to 0.6 wt %; or |
| | from 0.2 to 0.5 wt %; or |
| | from 0.3 to 0.5 wt %. |
| from 0.03 to 5 wt % | from 0.01 to 10 wt %; or |
| | from 0.02 to 7 wt %; or |
| | from 0.03 to 5 wt %; or |
| | from 0.05 to 3 wt %; or |
| | from 0.08 to 2 wt %; or |
| | from 0.1 to 1 wt %; or |
| | from 0.2 to 0.8 wt %; or |
| | from 0.2 to 0.6 wt %; or |
| | from 0.2 to 0.5 wt %; or |
| | from 0.3 to 0.5 wt %. |
| from 0.05 to 3 wt % | from 0.01 to 10 wt %; or |
| | from 0.02 to 7 wt %; or |
| | from 0.03 to 5 wt %; or |
| | from 0.05 to 3 wt %; or |
| | from 0.08 to 2 wt %; or |
| | from 0.1 to 1 wt %; or |
| | from 0.2 to 0.8 wt %; or |
| | from 0.2 to 0.6 wt %; or |
| | from 0.2 to 0.5 wt %; or |
| | from 0.3 to 0.5 wt %. |
| from 0.08 to 2 wt % | from 0.01 to 10 wt %; or |
| | from 0.02 to 7 wt %; or |
| | from 0.03 to 5 wt %; or |
| | from 0.05 to 3 wt %; or |
| | from 0.08 to 2 wt %; or |
| | from 0.1 to 1 wt %; or |
| | from 0.2 to 0.8 wt %; or |
| | from 0.2 to 0.6 wt %; or |
| | from 0.2 to 0.5 wt %; or |
| | from 0.3 to 0.5 wt %. |
| from 0.1 to 1 wt % | from 0.01 to 10 wt %; or |
| | from 0.02 to 7 wt %; or |
| | from 0.03 to 5 wt %; or |
| | from 0.05 to 3 wt %; or |
| | from 0.08 to 2 wt %; or |
| | from 0.1 to 1 wt %; or |
| | from 0.2 to 0.8 wt %; or |
| | from 0.2 to 0.6 wt %; or |
| | from 0.2 to 0.5 wt %; or |
| | from 0.3 to 0.5 wt %. |
| from 0.2 to 0.8 wt % | from 0.01 to 10 wt %; or |
| | from 0.02 to 7 wt %; or |
| | from 0.03 to 5 wt %; or |
| | from 0.05 to 3 wt %; or |
| | from 0.08 to 2 wt %; or |
| | from 0.1 to 1 wt %; or |
| | from 0.2 to 0.8 wt %; or |
| | from 0.2 to 0.6 wt %; or |
| | from 0.2 to 0.5 wt %; or |
| | from 0.3 to 0.5 wt %. |
| from 0.2 to 0.6 wt % | from 0.01 to 10 wt %; or |
| | from 0.02 to 7 wt %; or |
| | from 0.03 to 5 wt %; or |
| | from 0.05 to 3 wt %; or |
| | from 0.08 to 2 wt %; or |
| | from 0.1 to 1 wt %; or |
| | from 0.2 to 0.8 wt %; or |
| | from 0.2 to 0.6 wt %; or |
| | from 0.2 to 0.5 wt %; or |
| | from 0.3 to 0.5 wt %. |
| from 0.2 to 0.5 wt % | from 0.01 to 10 wt %; or |
| | from 0.02 to 7 wt %; or |
| | from 0.03 to 5 wt %; or |
| | from 0.05 to 3 wt %; or |
| | from 0.08 to 2 wt %; or |
| | from 0.1 to 1 wt %; or |
| | from 0.2 to 0.8 wt %; or |
| | from 0.2 to 0.6 wt %; or |
| | from 0.2 to 0.5 wt %; or |
| | from 0.3 to 0.5 wt %. |
| from 0.3 to 0.5 wt %. | from 0.01 to 10 wt %; or |
| | from 0.02 to 7 wt %; or |
| | from 0.03 to 5 wt %; or |
| | from 0.05 to 3 wt %; or |
| | from 0.08 to 2 wt %; or |
| | from 0.1 to 1 wt %; or |
| | from 0.2 to 0.8 wt %; or |
| | from 0.2 to 0.6 wt %; or |
| | from 0.2 to 0.5 wt %; or |
| | from 0.3 to 0.5 wt %. |

Methyl Cellulose Containing Liquid Formulations

| | |
|---|---|
| xanthan gum | methyl cellulose |

based on weight of the liquid formulation

| | |
|---|---|
| from 0.01 to 10 wt % | from 0.01 to 10 wt %; or |
| | from 0.02 to 7 wt %; or |
| | from 0.03 to 5 wt %; or |
| | from 0.05 to 3 wt %; or |
| | from 0.08 to 2 wt %; or |
| | from 0.1 to 1 wt %; or |
| | from 0.2 to 0.8 wt %; or |
| | from 0.2 to 0.6 wt %; or |
| | from 0.2 to 0.5 wt %; or |
| | from 0.3 to 0.5 wt %. |
| from 0.02 to 7 wt % | from 0.01 to 10 wt %; or |
| | from 0.02 to 7 wt %; or |
| | from 0.03 to 5 wt %; or |
| | from 0.05 to 3 wt %; or |
| | from 0.08 to 2 wt %; or |
| | from 0.1 to 1 wt %; or |
| | from 0.2 to 0.8 wt %; or |
| | from 0.2 to 0.6 wt %; or |
| | from 0.2 to 0.5 wt %; or |
| | from 0.3 to 0.5 wt %. |
| from 0.03 to 5 wt % | from 0.01 to 10 wt %; or |
| | from 0.02 to 7 wt %; or |
| | from 0.03 to 5 wt %; or |
| | from 0.05 to 3 wt %; or |
| | from 0.08 to 2 wt %; or |
| | from 0.1 to 1 wt %; or |
| | from 0.2 to 0.8 wt %; or |
| | from 0.2 to 0.6 wt %; or |
| | from 0.2 to 0.5 wt %; or |
| | from 0.3 to 0.5 wt %. |
| from 0.05 to 3 wt % | from 0.01 to 10 wt %; or |
| | from 0.02 to 7 wt %; or |
| | from 0.03 to 5 wt %; or |
| | from 0.05 to 3 wt %; or |
| | from 0.08 to 2 wt %; or |
| | from 0.1 to 1 wt %; or |
| | from 0.2 to 0.8 wt %; or |
| | from 0.2 to 0.6 wt %; or |
| | from 0.2 to 0.5 wt %; or |
| | from 0.3 to 0.5 wt %. |
| from 0.08 to 2 wt % | from 0.01 to 10 wt %; or |
| | from 0.02 to 7 wt %; or |
| | from 0.03 to 5 wt %; or |
| | from 0.05 to 3 wt %; or |
| | from 0.08 to 2 wt %; or |
| | from 0.1 to 1 wt %; or |
| | from 0.2 to 0.8 wt %; or |
| | from 0.2 to 0.6 wt %; or |
| | from 0.2 to 0.5 wt %; or |
| | from 0.3 to 0.5 wt %. |
| from 0.1 to 1 wt % | from 0.01 to 10 wt %; or |
| | from 0.02 to 7 wt %; or |
| | from 0.03 to 5 wt %; or |
| | from 0.05 to 3 wt %; or |
| | from 0.08 to 2 wt %; or |
| | from 0.1 to 1 wt %; or |
| | from 0.2 to 0.8 wt %; or |
| | from 0.2 to 0.6 wt %; or |
| | from 0.2 to 0.5 wt %; or |
| | from 0.3 to 0.5 wt %. |
| from 0.2 to 0.8 wt % | from 0.01 to 10 wt %; or |
| | from 0.02 to 7 wt %; or |
| | from 0.03 to 5 wt %; or |
| | from 0.05 to 3 wt %; or |
| | from 0.08 to 2 wt %; or |
| | from 0.1 to 1 wt %; or |
| | from 0.2 to 0.8 wt %; or |
| | from 0.2 to 0.6 wt %; or |
| | from 0.2 to 0.5 wt %; or |
| | from 0.3 to 0.5 wt %. |
| from 0.2 to 0.6 wt % | from 0.01 to 10 wt %; or |
| | from 0.02 to 7 wt %; or |
| | from 0.03 to 5 wt %; or |
| | from 0.05 to 3 wt %; or |
| | from 0.08 to 2 wt %; or |
| | from 0.1 to 1 wt %; or |
| | from 0.2 to 0.8 wt %; or |
| | from 0.2 to 0.6 wt %; or |
| | from 0.2 to 0.5 wt %; or |
| | from 0.3 to 0.5 wt %. |
| from 0.2 to 0.5 wt % | from 0.01 to 10 wt %; or |
| | from 0.02 to 7 wt %; or |
| | from 0.03 to 5 wt %; or |
| | from 0.05 to 3 wt %; or |
| | from 0.08 to 2 wt %; or |
| | from 0.1 to 1 wt %; or |
| | from 0.2 to 0.8 wt %; or |
| | from 0.2 to 0.6 wt %; or |
| | from 0.2 to 0.5 wt %; or |
| | from 0.3 to 0.5 wt %. |
| from 0.3 to 0.5 wt %. | from 0.01 to 10 wt %; or |
| | from 0.02 to 7 wt %; or |
| | from 0.03 to 5 wt %; or |
| | from 0.05 to 3 wt %; or |
| | from 0.08 to 2 wt %; or |
| | from 0.1 to 1 wt %; or |
| | from 0.2 to 0.8 wt %; or |
| | from 0.2 to 0.6 wt %; or |
| | from 0.2 to 0.5 wt %; or |
| | from 0.3 to 0.5 wt %. |

One type of liquid formulation comprises:

(i) a mixed metal compound containing at least one trivalent metal selected from iron (III) and aluminium and at least one divalent metal selected from of magnesium, iron, zinc, calcium, lanthanum and cerium, which is optionally fermagate;

(ii) xanthan gum in an amount of from 0.3 to 0.5 wt % based on the total liquid formulation; and

(iii) locust bean gum in an amount of from 0.3 to 0.5 wt % based on the total liquid formulation;

wherein the liquid formulation has been irradiated with ionising radiation in an amount of at least 4 kGy, such as from 4 to 10 kGy, such as at least 6 kGy, or such as from 6 to 10 kGy.

The liquid formulation may contain one or more further components. In one aspect, the liquid formulation is a pharmaceutical composition and further comprises (iv) one or more pharmaceutically acceptable adjuvants, excipients, diluents or carriers.

In one aspect the liquid formulation is substantially free of a wetting agent. Many insoluble drugs require wetting agents, e.g. to disperse the drug, or antifoaming agents, to prevent the inclusion of air bubbles in the formulation. The exclusion of a wetting agent is optionally when the mixed metal compound has a magnesium iron ratio between 1.5 and 2.5 and contains carbonate anions. By “substantially free of a wetting agent” it is meant the liquid formulation contains wetting agents in an amount of no greater than 10 wt %, or in an amount of no greater than 1 wt %, or in an amount of no greater than 0.5 wt %, or in an amount of no greater than 0.3 wt %, or in an amount of no greater than 0.22 wt %, or in an amount of no greater than 0.1 wt %, or in an amount of no greater than 0.05 wt %, or in an amount of no greater than 0.02 wt %, or in an amount of no greater than 0.01 wt %, or in an amount of no greater than 0.005 wt %, or in an amount of no greater than 0.001 wt %, or in an amount of no greater than 0.0001 wt %, or in an amount which is not measurable based on weight of the liquid formulation.

Another aspect to the liquid formulation is the combination of excipients has the effect of preventing any sensation of ‘grittiness’, due to the mixed metal compound component, in the mouth.

Sachets are a convenient form of container for single dose formulations, including liquid formulations, with the further advantage that the packaging material can be selected to withstand irradiation. Sachets can be selected which are suitable for single use only to avoid the need for prolonged in use microbial stability formulations; this because the use of preservatives are prohibitive in combinations with mixed metal compounds. Alternatively, the raw materials may be irradiated, however sources of microbial and bacterial contamination must be eliminated from the subsequent formulation make up and packaging stages to ensure sterility. This route is therefore less preferred, although still contemplated to be within the scope of the methods of making liquid formulations for use herein.

The liquid formulations can irradiated within 5 days after preparation of the formulation, or within 2 days, or within 1 day, or immediately after preparation of the liquid formulation. It will be appreciated to one skilled in the art that initial microbial and fungal content of the raw materials and the cleanliness of the formulation preparation (i.e. prior to irradiation) is such as to minimise microbial and fungal contamination.

Polymers for use in packaging, such as sachets, which show tolerance to irradiation include polystyrene, polyethylene, polyesters, polysulfone, polycarbonates, polyurethane, PVC, Silicone, Nylon, Polypropylene (irradiation grades) and Fluoroplastics.

Where metallic foils are used as materials of construction for sachets, care must be taken when selecting materials to avoid e.g. leaching into or reaction with the sachet contents or should be coated with a suitable polymer to avoid leaching.

Optional embodiments include a liquid formulation based on an combination of xanthan gum (0.35% w/v) and locust bean gum (0.35% w/v) which is preserved by irradiation at a dose level (6 kGy). Another embodiment is a liquid formulation based on a combination of PVP (0.5% w/v) and xanthan gum (1.0% w/v) which is preserved by irradiation at a dose level (8 kGy). Another embodiment is a liquid formulation based on a combination of methyl cellulose with xanthan gum which is preserved by irradiation at a dose level (10 kGy). Each of these formulations is contemplated to optionally include sorbitol at a concentration of 6% w/v.

Liquid formulation with a yield stress have the theoretical ability to suspend solids within the liquid formulation indefinitely. Because the liquid formulation must be able to be handled during manufacture and poured and/or squeezed from a container during use, the yield value should not be more than 19 Pa. Of course, if the formulation is to be squeezed from a sachet, for example, higher yield stress values might be acceptable but are optionally limited to less than 30 Pa (to maintain patient palatability and or texture).

The liquid formulation should be easy to mix, pour or squeeze and swallow, while maintaining the mixed metal compound in suspension and stable upon storage. Consequently there is a need for a formulation that is of low viscosity at high shear and of high viscosity at low shear. Thus an optimum range of yield stress and a low viscosity at high shear and of high viscosity at low shear exists. An optimum yield stress for the liquid formulation from 0.5 to approximately 19 Pa is contemplated.

### Phosphate Binding

Phosphate binding capacity can be determined by the following method: 40 mmoles/liter Sodium Phosphate solution (pH 4) is prepared and treated with the phosphate-binder. The filtered solution of the treated phosphate solution is then diluted and analyzed by ICP-OES for phosphorus content.

Reagents used for this method are: Sodium Dihydrogen Phosphate Monohydrate (BDH, AnalaR™ grade), 1M hydrochloric acid, AnalaR™ water), standard phosphorous solution (10,000 pg/ml, Romil Ltd), sodium chloride (BDH).

Specific apparatus used are: Rolling hybridization incubator or equivalent (Grant Boekal HIW7), 10 ml blood collection tubes, Reusable Nalgene screw cap tubes (30 ml/50 ml), 10 ml disposable syringes, 0.45 pm single use syringe filter, ICP-OES (inductively coupled plasma—optical emission spectrometer).

Phosphate solution is prepared by weighing 5.520 g (+/−0.001 g) of sodium di-hydrogen phosphate followed by addition of some AnalaR™ water and transferring to a lift volumetric flask.

To the 1 liter volumetric flask is then added 1 M HCI drop-wise to adjust the pH to pH 4 (+/−0.1) mixing between additions. The volume is then accurately made up to one liter using AnalaR™ water and mixed thoroughly.

NaCl solution is prepared by accurately weighing out 5.85 g (+/−0.02 g) of NaCl and quantitatively transferring into a 1 liter volumetric flask after which the volume is made up with AnalaR™ water and mixed thoroughly.

Calibration Standards are prepared by pipetting into 100 ml volumetric flasks the following solutions:

| Flask No. | 1 | | 2 | 3 | 4 |
|---|---|---|---|---|---|
| Identification | Blank | | Std 1 | Std 2 | Std 3 |
| NaCI solution | 10 | ml | 10 ml | 10 ml | 10 ml |
| 10000 ppm P Std | 0 | ml | 4 ml | 2 ml | 1 ml |
| | | | (400 ppm) | (200 ppm) | (100 ppm) |

The solutions are then made up to volume with AnalaR™ water and thoroughly mixed. These solutions are then used as calibration standards for the ICP-OES apparatus. The phosphate binder samples are then prepared in accordance with the procedure described hereafter and measured by ICP-OES. The ICP-OES results are initially expressed as ppm but can be converted to mmol using the equation: mmol=(reading ICP-OES in ppm/molecular weight of the analyte)×4 (dilution factor).

Aliquots of each test sample, each aliquot containing 0.5 g of the phosphate binder, are placed into 30 ml screw top Nalgene tubes. If the test sample is a unit dose comprising 0.5 g of the phosphate binder, it may be used as such. All samples are prepared in duplicate. 12.5 ml aliquots of the Phosphate solution are pipetted into each of the screw top tubes containing the test samples and the screw cap fitted. The prepared tubes are then placed into the roller incubator pre heated to 37° C. and rotated at full speed for a fixed time such as 30 minutes (other times may be used as shown in the Examples). The samples are subsequently removed from the roller incubator, filtered through a 0.45 pm syringe filter, and 2.5 ml of filtrate transferred into a blood collection tube. 7.5 ml of AnalaR™ water is pipetted into each 2.5 ml aliquot, and mixed thoroughly. The solutions are then analyzed on the ICP-OES.

The phosphate binding capacity is determined by: phosphate binding (%)=100−(T/S×100)

where

T=Analyte value for phosphate in solution after reaction with phosphate binder.

S=Analyte value for phosphate in solution before reaction with phosphate binder.

In accordance with embodiments, the mixed metal compounds can provide a phosphate binding capacity as measured by the above method of at least 30% after 30 minutes, at least 30% after 10 minutes, at least 30% after 5 minutes. In embodiments, the water-insoluble inorganic solid mixed metal compound can be formulated into tablets and have a phosphate binding capacity as measured by the above method of at least 40% after 30 minutes, at least 30% after 10 minutes, at least 30% after 5 minutes, at least 50% after 30 minutes, at least 30% after 10 minutes, or at least 30% after 5 minutes.

The pH of the phosphate binding measurement may be varied by use of addition of either 1M HCI or NaOH solution. The measurement may then be used to assess the phosphate binding capacity at varying pH values.

In embodiments, the water-insoluble inorganic solid mixed metal compound can have a phosphate binding capacity at a pH from 3 to 6, at a pH from 3 to 9, at a pH from 3 to 10, at a pH from 2 to 10, as measured by the above method, of at least 30% after 30 minutes, at least 30% after 10 minutes, at least 30% after 5 minutes.

In embodiments, the water-insoluble inorganic solid mixed metal compound can have a phosphate binding capacity at a pH from 3 to 4, from 3 to 5, from 3 to 6 as measured by the above method of at least 40% after 30 minutes, at least 40% after 10 minutes, at least 40% after 5 minutes.

In embodiments, the water-insoluble inorganic solid mixed metal compound can have a phosphate binding capacity at a pH from 3 to 4, from 3 to 5, from 3 to 6, as measured by the above method, of at least 50% after 30 minutes, at least 50% after 10 minutes, at least 50% after 5 minutes.

It will be understood that it is desirable to have high phosphate binding capability over as broad a pH range as possible.

An alternate method of expressing phosphate binding capacity using the method described above is to express the phosphate bound by the binder as mmol of Phosphate bound per gram of binder.

Using this description for phosphate binding, suitably, the water-insoluble inorganic solid mixed metal compounds can have in embodiments a phosphate binding capacity at a pH from 3 to 6, at a pH from 3 to 9, at a pH from 3 to 10, at a pH from 2 to 10 as measured by the above method of at least 0.3 mmol/g after 30 minutes, at least 0.3 mmol/g after 10 minutes, at least 0.3 mmol/g after 5 minutes. In embodiments, the water-insoluble inorganic solid mixed metal compound can have a phosphate binding capacity at a pH from 3 to 4, 3 to 5, from 3 to 6 as measured by the above method of at least 0.4 mmol/g after 30 minutes, at least 0.4 mmol/g after 10 minutes, at least 0.4 mmol/g after 5 minutes. n embodiments, the water-insoluble inorganic solid mixed metal compound can have a phosphate binding capacity at a pH from 3 to 4, from 3 to 5, from 3 to 6 as measured by the above method of at least 0.5 mmol/g after 30 minutes, at least 0.5 mmol/g after 10 minutes, at least 0.5 mmol/g after 5 minutes.

The Test Methods referred to above are described below.

### Test Method 1 XRF Analysis

XRF analysis can be performed by using a Philips PW2400 Wavelength Dispersive XRF Spectrometer. The sample is fused with 50:50 lithium tetra/metaborate (high purity) and presented to the instrument as a glass bead. All reagents are analytical grade or equivalent unless specified. AnalaR™ water, Lithium tetraborate 50% metaborate 50% flux (high purity grade ICPH Fluore-X 50). A muffle furnace capable of 1025° C., extended tongs, hand tongs, Pt/5% Au casting tray and Pt/5%/Au dish are used. 1.5 g (+/−0.0002 g) of sample and 7.5000 g (+/−0.0002 g) of tetra/metaborate is accurately weighed out into a Pt/5%/Au dish. The two constituents are lightly mixed in the dish using a spatula, prior to placement in the furnace preset to 1025° C. for 12 minutes. The dish is agitated at 6 minutes and 9 minutes to ensure homogeneity of the sample. Also at 9 minutes the casting tray is placed in the furnace to allow for temperature equilibration. After 12 minutes the molten sample is poured into the casting tray, which is removed from the furnace and allowed to cool. The bead composition is determined using the spectrophotometer.

The XRF method can be used to determine the Al, Fe, Mg, Na and total sulphate content of the mixed metal compound, as well as the M^{II} to M^{III} ratio.

### Test Method 2 X-Ray Diffraction (XRD) Measurements

Powder X-ray diffraction (XRD) data are collected from 2-70° 2θ on a Philips PW 1800 automatic powder X-ray diffractometer using copper K alpha radiation generated at 40 kV and 55 mA, a 0.02° 2θ step size with a 4 second per step count time. An automatic divergence slit giving an irradiated sample area of 15×20 mm is used, together with a 0.3 mm receiving slit and a diffracted beam monochromator.

The approximate volume average crystallite size can be determined from the width, at half peak height, of the powder X-ray diffraction peak at about 11.5° 2θ (the peak is typically in the range 8 to 15 degrees 2 theta for hydrotalcite type materials) using the relationship given in the table below which is derived using the Scherrer equation. The contribution to the peak width from instrument line broadening is 0.15 degrees, determined by measuring the width of the peak at approximately 21.4° 2θ of a sample of LaB6 (NIST SRM 660) under the same conditions.

### XRD Peak Width Conversion to Crystallite Size Using the Scherrer Equation

| Peak width | | D - |
|---|---|---|
| FWHM B | B(measured) - | Calculated |
| (measured) | b (instrument) | crystallite |
| (°2Θ) | (°2Θ) | size (Å) |
| 0.46 | 0.31 | 258 |
| 0.47 | 0.32 | 250 |
| 0.48 | 0.33 | 242 |
| 0.49 | 0.34 | 235 |
| 0.50 | 0.35 | 228 |
| 0.51 | 0.36 | 222 |
| 0.52 | 0.37 | 216 |
| 0.53 | 0.38 | 210 |
| 0.54 | 0.39 | 205 |
| 0.55 | 0.40 | 200 |
| 0.56 | 0.41 | 195 |
| 0.57 | 0.42 | 190 |
| 0.58 | 0.43 | 186 |
| 0.59 | 0.44 | 181 |
| 0.60 | 0.45 | 177 |
| 0.61 | 0.46 | 174 |
| 0.62 | 0.47 | 170 |
| 0.63 | 0.48 | 166 |
| 0.64 | 0.49 | 163 |
| 0.65 | 0.50 | 160 |
| 0.66 | 0.51 | 157 |
| 0.67 | 0.52 | 154 |
| 0.68 | 0.53 | 151 |
| 0.69 | 0.54 | 148 |
| 0.70 | 0.55 | 145 |
| 0.71 | 0.56 | 143 |
| 0.72 | 0.57 | 140 |
| 0.73 | 0.58 | 138 |
| 0.74 | 0.59 | 135 |
| 0.75 | 0.60 | 133 |
| 0.76 | 0.61 | 131 |
| 0.77 | 0.62 | 129 |
| 0.78 | 0.63 | 127 |
| 0.79 | 0.64 | 125 |
| 0.80 | 0.65 | 123 |
| 0.81 | 0.66 | 121 |
| 0.82 | 0.67 | 119 |
| 0.83 | 0.68 | 117 |
| 0.84 | 0.69 | 116 |
| 0.85 | 0.70 | 114 |
| 0.86 | 0.71 | 112 |
| 0.87 | 0.72 | 111 |
| 0.88 | 0.73 | 109 |
| 0.89 | 0.74 | 108 |
| 0.90 | 0.75 | 106 |
| 0.91 | 0.76 | 105 |
| 0.92 | 0.77 | 104 |
| 0.93 | 0.78 | 102 |
| 0.94 | 0.79 | 101 |
| 0.95 | 0.80 | 100 |
| 0.96 | 0.81 | 99 |
| 0.97 | 0.82 | 97 |
| 0.98 | 0.83 | 96 |
| 0.99 | 0.84 | 95 |
| 1.00 | 0.85 | 94 |
| 1.01 | 0.86 | 93 |
| 1.02 | 0.87 | 92 |
| 1.03 | 0.88 | 91 |
| 1.04 | 0.89 | 90 |
| 1.05 | 0.90 | 89 |
| 1.06 | 0.91 | 88 |
| 1.07 | 0.92 | 87 |
| 1.08 | 0.93 | 86 |
| 1.09 | 0.94 | 85 |
| 1.10 | 0.95 | 84 |
| 1.11 | 0.96 | 83 |
| 1.12 | 0.97 | 82 |
| 1.13 | 0.98 | 81 |
| 1.14 | 0.99 | 81 |
| 1.15 | 1.00 | 80 |
| 1.16 | 1.01 | 79 |
| 1.17 | 1.02 | 78 |
| 1.18 | 1.03 | 78 |
| 1.19 | 1.04 | 77 |
| 1.20 | 1.05 | 76 |
| 1.21 | 1.06 | 75 |
| 1.22 | 1.07 | 75 |
| 1.23 | 1.08 | 74 |
| 1.24 | 1.09 | 73 |
| 1.25 | 1.10 | 73 |
| 1.26 | 1.11 | 72 |
| 1.27 | 1.12 | 71 |
| 1.28 | 1.13 | 71 |
| 1.29 | 1.14 | 70 |
| 1.30 | 1.15 | 69 |
| 1.31 | 1.16 | 69 |

The values in the table above were calculated using the Scherrer equation:

D=K*λ/β*cos Θ Equation 1

### Where:

D=crystallite size (A)
K=shape factor
λ=wavelength of radiation used (in Å)
β=peak width measured as FWHM (full width at half maximum height) and corrected for instrument line broadening (expressed in radians)
Θ=the diffraction angle (half of peak position 2Θ, measured in radians)

### Shape Factor

This is a factor for the shape of the particle, typically between 0.8-1.0, a value of 0.9 is used.

### Wavelength of Radiation

This is the wavelength of the radiation used. For copper K alpha radiation the value used is 1.54056 Å.

### Peak Width

The width of a peak is the sum of two sets of factors: instrumental and sample.

The instrumental factors are typically measured by measuring the peak width of a highly crystalline sample (very narrow peaks). Since a highly crystalline sample of the same material is not available, LaB6 has been used. For the current measurements an instrument value of 0.15 degrees is used.

Thus for the most accurate measure of crystallite size using the Scherrer equation, the peak width due to instrumental factors should be subtracted from the measured peak width i.e.:

β=B_{(measured)}−b₍ᵢₙₛₜᵣᵤₘₑₙₜₐₗ₎

The peak width is then expressed in radians in the Scherrer equation.

The peak width (as FWHM) is measured by fitting of a parabola or another suitable method to the peak after subtraction of a suitable background.

### Peak Position

A value of 11.5° 2Θ has been used giving a diffraction angle of 5.75°, corresponding to 0.100 radians.

### Test Method 3 Phosphate Binding Capacity and Mg Release

Phosphate buffer (pH=4) is prepared by weighing 5.520 g (+/−0.001 g) of sodium di-hydrogen phosphate followed by addition of AnalaR™ water and transferring to a 1 ltr volumetric flask.

To the 1 liter volumetric flask is then added 1 M HCl drop-wise to adjust the pH to pH 4 (+/−0.1) mixing between additions. The volume is then accurately made up to 1 ltr using AnalaR™ water and mixed thoroughly.

0.5 g (+/−0.005 g) of each sample is added to a volumetric flask (50 ml) containing 40 mM phosphate buffer solution (12.5 ml) at 37.5° C. in a Grant OLS 200 Orbital shaker. All samples are prepared in duplicate. The vessels are agitated in the orbital shaker for 30 minutes. The solution is then filtered using a 0.45 μm syringe filter. 2.5 cm³ aliquots of supernatant are pipetted of the supernatant and transferred into fresh blood collection tubes. 7.5 cm³ of AnalaR™ water are pipetted to each 2.5 cm³ aliquot and the screw cap fitted and mixed thoroughly. The solutions are then analyzed on a calibrated ICP-OES.

The phosphate binding capacity is determined by:

Phosphate binding (mmol/g)=S_{P} (mmol/l)−T_{P} (mmol/l)/W (g/l)

where:
T_{P}=Analyte value for phosphate in the phosphate solution after reaction with phosphate binder=solution P (mg/l)*4/30.97;
S_{P}=Analyte value for phosphate in the phosphate solution before reaction with phosphate binder; and
W=concentration binder (g/l) used in test method (i.e. 0.4 g/10 cm³=40 g/l).

Magnesium release is determined by:

Magnesium release (mmol/g)=T_{Mg} (mmol/l)−S_{Mg} (mmol/l)/W (g/l)

where:
T_{Mg}=Analyte value for magnesium in the phosphate solution after reaction with phosphate binder=solution Mg (mg/l)*4/24.31; and
S_{Mg}=Analyte value for magnesium in the phosphate solution before reaction with phosphate binder.

Fe release is not reported as the amount of iron released from the compound is too small and below detection limit.

### Test Method 4 Phosphate Binding and Magnesium Release in Food Slurry

MCT peptide2+, food supplement (SHS International) is mixed to form a slurry of 20% (w/v) in 0.01 M HCl. Separate aliquots of 0.05 g dry compound are mixed with 5 cm³ of the food slurry and constantly agitated for 30 minutes at room temperature. A 3 cm³ aliquot is removed and centrifuged at 4000 rpm for 10 minutes, and the phosphate and magnesium in solution are measured.

### Test Method 5 Sulphate Determination

Sulphite (SO₃) is measured in the compound by XRF measurement (Test Method 1) and expressed as total sulphate (SO₄) according to:

Total SO₄(wt %)=(SO₃)×96/80.

Total SO₄ (mole)=total SO₄ (wt %)/molecular weight SO4

Sodium Sulphate (Soluble Form of Sulphate Present in the Compound)

Na₂O is measured in the compound by XRF measurement (Test Method 1).

It is assumed that the Na₂O is associated with the more soluble form of SO₄ in the form of Na₂SO₄ present in the compound.

Consequently, the number of mole Na₂O is assumed equal to that of soluble form of sulphate and is therefore calculated as:

soluble SO₄ (mole)=Na₂O (mole)=wt % Na₂O/molecular weight Na₂O

Interlayer sulphate (insoluble form of sulphate present in the compound also referred to as bound sulphate).

The interlayer sulphate is calculated according to:

interlayer SO₄ (mole)=total SO₄ (mole)−soluble SO₄ (mole)

interlayer SO₄ (wt %)=interlayer SO₄ (mole)×molecular weight SO₄.

### Test Method 6 Carbon Content Analysis by the Leco Method

This method is used to determine the levels of carbon content (indicative of the presence of the carbonate anion present in the mixed metal compound).

A sample of known mass is combusted at around 1350° C. in a furnace in a pure oxygen atmosphere. Any carbon in the sample is converted to CO₂ which is passed through a moisture trap before being measured by an infra-red detector. By comparing against a standard of known concentration, the carbon content of the sample can be found. A Leco SC-144DR carbon and Sulphur Analyser, with oxygen supply, ceramic combustion boats, boat lance and tongs is used. 0.2 g (+/−0.01 g) of sample is weighed into a combustion boat. The boat is then placed into the Leco furnace and the carbon content analyzed. The analysis is performed in duplicate.

The % C is determined by:

% C(sample)=(% C₁+% C₂)/2

Where C₁ and C₂ are individual carbon results.

### Test Method 7 Particle Size Distribution (PSD) by Lasentech

In process particle size distribution in the slurry can be measured using a Lasentech probe. The d50 average particle size, is obtained as part of this analytical technique.

### Test Method 8 Moisture Content

The moisture content of mixed metal compound is determined from the loss of weight (LOD) following drying at 105° C. for four hours at ambient pressure in a laboratory oven.

### Test Method 9 Surface Area and Pore Volume (Nitrogen Method —N₂)

Surface area and pore volume measurements are obtained using nitrogen gas adsorption over a range of relative pressures using a Micromeritics Tristar ASAP 3000. The samples are outgassed under vacuum for 4 hours at 105° C. before the commencement of measurements. Typically a vacuum of <70 mTorr is obtained after outgassing.

Surface areas re calculated by the application of Brunauer, Emmett and Teller (BET) theory using nitrogen adsorption data obtained in the relative pressure range of 0.08 to 0.20 P/Po.

Pore volume is obtained from the desorption loop of the nitrogen adsorption isotherm, using the volume of gas adsorbed at a relative pressure (P/Po) of 0.98. The quantity of gas adsorbed at 0.98 relative pressure (in cc/g at STP) is converted to a liquid equivalent volume by multiplying by the density conversion factor of 0.0015468. This gives the reported pore volume figure in cm³/g.

P=partial vapor pressure of nitrogen in equilibrium with the sample at 77K.
Po=saturated pressure of nitrogen gas.

### Test Method 10 Pore Volume (Water Method)

Water Pore Volume

Aim

To fill internal pores of a sample (in powder form) with water such that, when all the pores are filled, the surface tension of the liquid causes the majority of the sample to form an aggregate which adheres to a glass jar on inversion of the jar.

Equipment

(1) Wide neck (30 mm) clear glass 120 cm³ powder jar with screw cap. Dimensions: Height 97 mm. Outer Diameter 50 mm. (Fisher part number BTF-600-080)
(2) 10 cm³ Grade A burette
(3) Deionized water
(4) Rubber bung 74 mm diameter top tapered to 67 mm. Overall height 49 mm.
(5) Calibrated 4 decimal place balance

### Procedure

(1) To a 5.00 g (±0.01) sample in the glass jar, add a 1 cm³ aliquot of water
(2) After this addition vigorously knock the bottom end of the sealed jar against the rubber bung 4 times.
(3) Using a sharp swing of the arm, flick the jar with the wrist to invert the jar and check the sample:
a. If the sample agglomerates and the majority (>50%) of the sample adheres to the jar this is the end point (go to results section below). If free water is observed with the sample, the end point has been exceeded and the test should be discarded and started again with a new sample.
b. If the sample dislodges from the jar (even if agglomeration is evident), add a further 0.1 cm³ of water and repeat steps (2) to (3) above until the end point is reached (3a)).

### Results

The water pore volume is calculated as follows:

Water Pore Volume (cm³/g)=Volume of water added (cm³)/Sample Weight (g).

### Test Method 11

### (a) Determination of Phosphate Binding Capacity and Soluble Magnesium/Iron Using Standard Method

40 mM Sodium Phosphate solution (pH 4) is prepared and treated with the phosphate-binder. The supernatant of the centrifuged phosphate-solution and binder mixture is then diluted and analyzed by ICP-OES for Fe, Mg and P content. The latter analysis technique is well known to those skilled in the art. ICP-OES is the acronym for inductively coupled plasma optical emission spectroscopy.

Reagents used for this method are: Sodium Dihydrogen Phosphate Monohydrate (Aldrich), 1M hydrochloric acid, AnalaR™ water, standard phosphorous solution (10.000 μg/ml, Romil Ltd), standard magnesium solution (10,000 μg/ml, Romil Ltd), standard iron solution (1.000 μg/ml), sodium chloride (BDH).

Specific apparatus are: centrifuge (Metier 2000E), blood-tube rotator (Stuart Scientific), minishaker (MS1), ICP-OES, blood collection tubes. Phosphate buffer (pH=4) is prepared by weighing 5.520 g (+/−0.001 g) of sodium di-hydrogen phosphate followed by addition of AnalaR™ water and transferring to a 1 ltr volumetric flask.

To the 1 ltr volumetric flask is then added 1 M HCI drop-wise to adjust the pH to pH 4 (+/−0.1) mixing between additions. The volume is then accurately made up to 1 liter using AnalaR™ water and mixed thoroughly.

0.4 g (+/−0.005 g) of each sample is weighed into blood collection tubes and placed in a holding rack. All samples are prepared in duplicate and temperature of solutions maintained at 20° C. 10 ml aliquots of the phosphate buffer were pipetted into each of the blood collection tubes containing the pre-weighed test materials and the screw cap fitted. The vessels are agitated over a minishaker for about ten seconds. The vessels are transferred onto a blood tube rotator and mixed for 30 minutes (+/−2 minutes). The vessels are then centrifuged at 3000 rpm and 20° C. for 5 minutes. The samples are removed from the centrifuge and 2.5 ml aliquots are pipetted of the supernatant and transferred into a fresh blood collection tubes. 7.5 ml of AnalaR™ water are pipetted to each 2.5 ml aliquot and the screw cap fitted and mixed thoroughly. The solutions are then analyzed on a calibrated ICP-OES.

The phosphate binding capacity is determined by:

Phosphate binding (mmol/g)=[S_{P} (mmol/l)−T_{P} (mmol/l)]/W (g/l)

where: Tₚ=Analyte value for phosphate in the phosphate solution after reaction with phosphate binder=solution P (mg/l)*4/30.97. Tₚ used in test method 11a and Tₚ¹ used instead of Tₚ for test method 11b, 11c;
Sp=Analyte value for phosphate in the phosphate solution before reaction with phosphate binder; and
W=concentration binder (g/l) used in test method (i.e. 0.4 g/10 ml in test method 11a=40 g/l)

Magnesium release is determined by:

Magnesium release (mmol/g)=[T_{Mg} (mmol/l)−S_{Mg} (mmol/l)]/W (g/l)

where: T_{Mg}=Analyte value for magnesium in the phosphate solution after reaction with phosphate binder=solution Mg (mg/l)*4/24.31. T_{Mg} used in test method 11a and T_{Mg}¹ used instead of T_{Mg} for test method 11b, and 11c; and
S_{Mg}=Analyte value for magnesium in the phosphate solution before reaction with phosphate binder.

Iron release is determined by:

Iron release (mmol/g)=[T_{Fe} (mmol/l)−S_{Fe} (mmol/l)]/W (g/l)

where: T_{Fe}=Analyte value for iron in the phosphate solution after reaction with phosphate binder=solution Fe (mg/l)*4/55.85. T_{Fe} used in test method 11a and T_{Fe}¹ used instead of T_{Fe} for test method 11b, 11c; and
S_{Fe}=Analyte value for iron in the phosphate solution before reaction with phosphate binder.

### (b) Determination of Phosphate Binding Capacity and Soluble Magnesium/Iron Using Representative Method at 0.4 g Phosphate Binder/10 ml.

The standard phosphate binding test Method 11 (a) involves the use of phosphate buffer adjusted to pH 4. The pH of this test can increase from pH 4 to approx 8.5-9 after addition of the mixed metal compounds. Test method 11 b can be used to determine the phosphate binding capacity using a more representative method of conditions under gastric conditions (lower pH value of 3) and by maintaining the pH at a constant value by the addition of 1M HCI during the phosphate binding, contrary to the standard phosphate binding test where the pH is allowed to rise during the phosphate binding.

The representative method (for measuring phosphate binding and magnesium- or iron-release) is maintained as per standard phosphate binding test Test Method 11 (a), i.e. 0.4 g of the phosphate binder is dispersed in 10 ml phosphate buffer. The temperature of solutions is 20° C. In order to monitor the pH, the sample is weighed into a Sterlin Jar. This jar is placed on a stirrer plate with stirrer placed in jar. The 10 ml of the phosphate buffer is added to the sample and the pH hereafter immediately monitored via a pH probe during 30 minutes and the pH is maintained at pH=3 using 1M HCI delivered via a Dosimat titrator. The total volume of acid added for pH adjustment should not exceed 61% of the total volume.

The phosphate binding and Mg⁻ and Fe⁻ release data of the representative method is then corrected for the dilution of phosphate or compound concentration due to acid addition (as phosphate binding and Mg⁻ and Fe⁻ release are measured from the difference between before and after the phosphate binding reaction) using the following formula, wherein V is the volume (ml) of 1 M HCl acid used for pH adjustment in the representative method:

Tₚ¹=Tₚ*(10 ml+V)/10 ml

T_{Mg}¹=T_{Mg}*(10 ml+V)/10 ml

T_{Fe}¹=T_{Fe}*(10 ml+V)/10 ml

wherein Tₚ=analyte concentration for phosphate after reaction with phosphate binder Tₚ¹=identical as Tₚ but with concentration corrected for dilution because of acid addition;
T_{Mg}=analyte concentration for magnesium after reaction with phosphate binder T_{Mg}¹=identical as T_{Mg} but with concentration corrected for dilution because of acid addition; and
T_{Fe}=analyte concentration for iron after reaction with phosphate binder T_{Fe}¹=identical as T_{Fe} but with concentration corrected for dilution because of acid addition.

After the 30 minutes phosphate binding, the slurry is transferred to a blood sample tube (approx 10 ml) and centrifuged for 5 minutes at 3000 RPM. Then as per standard phosphate binding Test Method 11 (a) 2.5 ml of the supernatant is diluted to 10 ml with AnalaR water in a separate collection tube, ready for analysis on the ICP.

### c) Determination of Phosphate Binding Capacity and Soluble Magnesium/Iron Using Representative Method at 0.2 g Phosphate Binder/10 ml.

Identical method to that described in method 11b but with 0.2 g phosphate binder/10 ml

### Test Method 14 Surface Area and Pore Volume (Nitrogen Method —N₂)

Surface area and pore volume measurements are obtained using nitrogen gas adsorption over a range of relative pressures using a Micromeritics Tristar ASAP 3000. The samples are outgassed under vacuum for 4 hours at 105° C. before the commencement of measurements. Typically a vacuum of <70 mTorr is obtained after outgassing.

Surface areas are calculated by the application of Brunauer, Emmett and Teller (BET) theory using nitrogen adsorption data obtained in the relative pressure range of 0.08 to 0.20 P/Po.

Pore volume is obtained from the desorption loop of the nitrogen adsorption isotherm, using the volume of gas adsorbed at a relative pressure (P/Po) of 0.98. The quantity of gas adsorbed at 0.98 relative pressure (in cc/g at STP) is converted to a liquid equivalent volume by multiplying by the density conversion factor of 0.0015468. This gives a reported pore volume figure in cm³/g.

P=partial vapour pressure of nitrogen in equilibrium with the sample at 77K.

Po=saturated pressure of nitrogen gas.

### EXAMPLES

The following examples are provided for illustration and are not intended to limit the scope of the invention.

### Example 1

Described below is an examination of the effect of fermagate and dietary phosphate on the severity of mineral bone disorder outcomes including serum phosphate, calcium, PTH, and FGF23, and of vascular calcification in an adenine rat model of CKD.

The studies described herein were carried out to determine if fermagate treatment compared to untreated control could impact VC in the adenine-induced CKD rat model with two methods of dietary phosphate delivery.

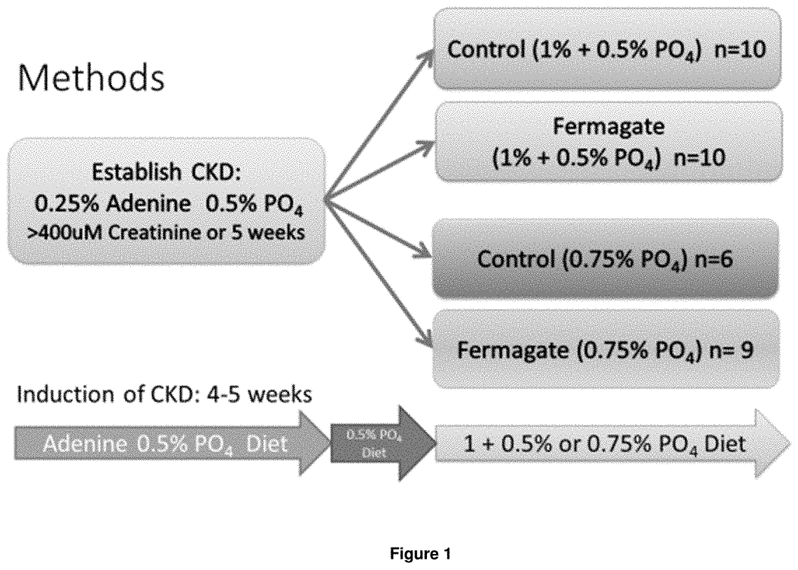

Male Sprague Dawley rats (15 weeks) were fed a 0.25% adenine, 0.5% phosphate (PO₄) diet to induce CKD (creatinine >250 uM) over 4-5 weeks, then fed 0.5% PO₄ without adenine diet. At 6 weeks CKD, two dietary PO₄ regimens were tested: moderate PO₄ (0.75% P) diet (5 g at 8 AM and 4 PM ±fermagate (FER n=9) untreated control (CON n=6), 10 grams diet overnight, or a combination of high and low PO₄ (1-0.5% P): high (1% P 5 g 8 AM, 4 PM ±fermagate) and 10 g low (0.5% P) PO₄ diet overnight (FER n=8, CON n=10) with the same amount of daily dietary PO₄. Serum calcium (Ca), magnesium (Mg), PO₄, FGF23, parathyroid hormone (PTH), vitamin D metabolome, and tissue Ca and PO₄ were determined. The method is graphically represented in FIG. 1.

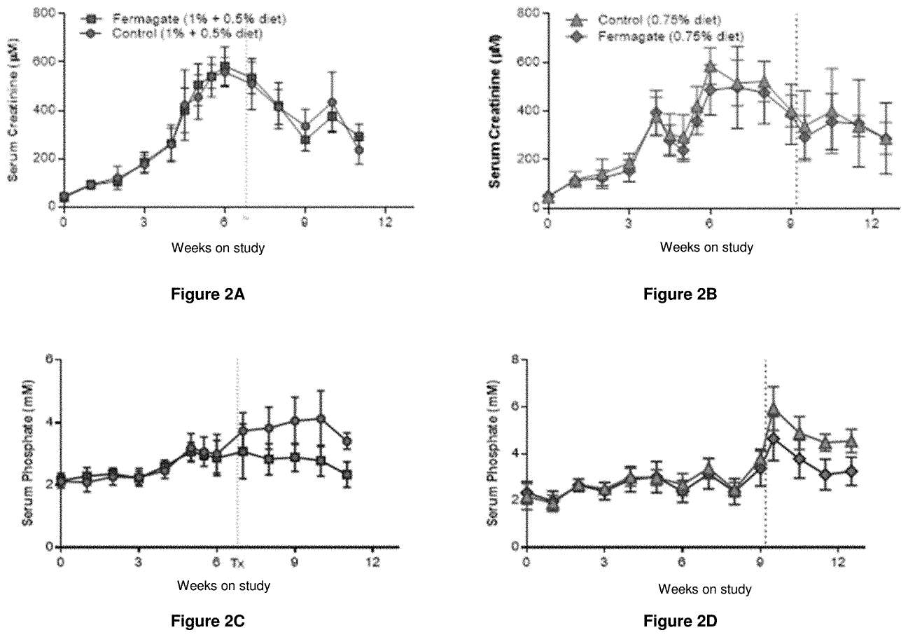

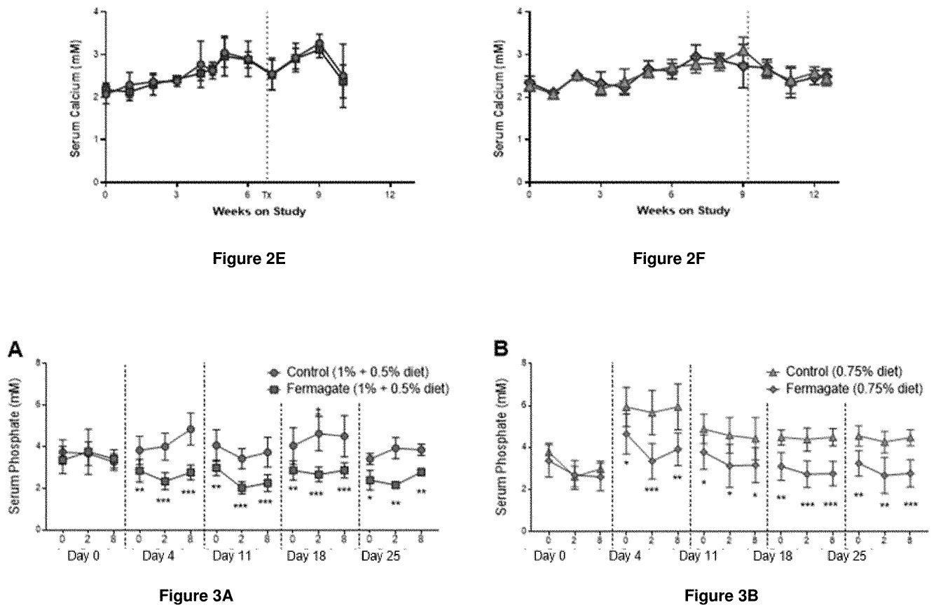

The results illustrated in FIG. 2 show that the Chronic Kidney Disease induction phenotype was similar between fermagate-treated and control animals. Creatinine and calcium profiles were not significantly different across the time courses for both experiments. Serum phosphate levels were similar until the change in dietary phosphate and start of fermagate (dotted line). After start of fermagate treatment and higher phosphate diet, hyperphosphatemia was present only in control.

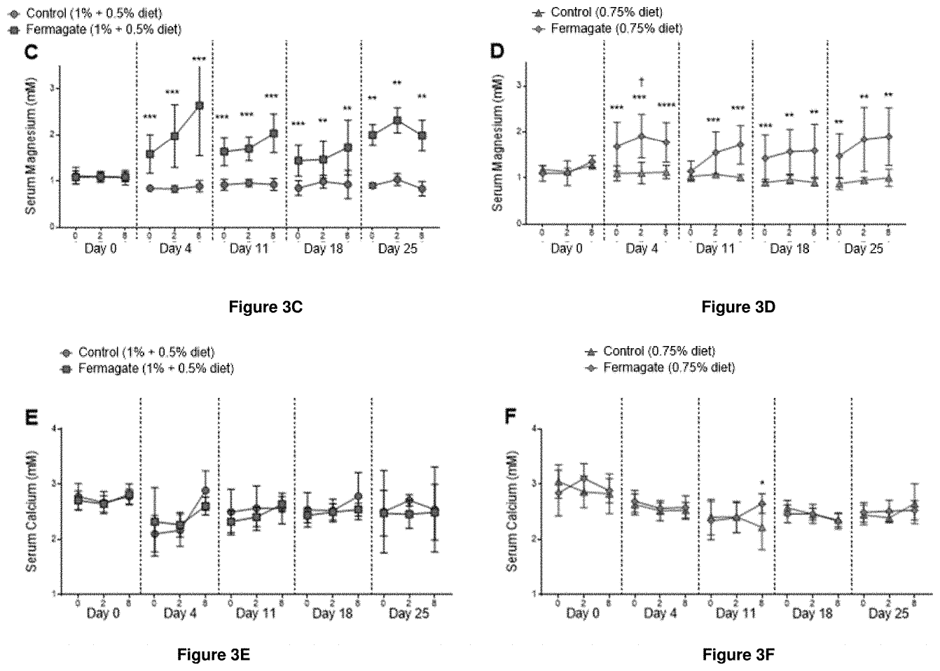

The results illustrated in FIG. 3 show that fermagate decreases serum phosphate while increasing magnesium. In both studies, fermagate increased serum Mg (203% 0.75% P, p<0.0001; 163% 0.5-1% P, p<0.0001, % control, 2-way ANOVA) and had lower levels of serum PO₄ (67% 0.75% P, p<0.001; 64% 0.5-1% P, p<0.001).

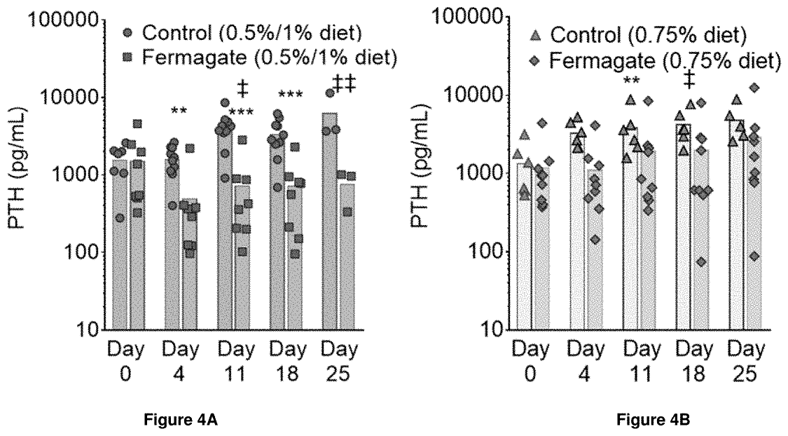

The results illustrated in FIG. 4 show that parathyroid hormone levels increased in the control animals, while PTH levels were surprisingly and significantly lower with fermagate treatment (31% 1+0.5%/1% P diet, p<0.001; 16% 0.75% diet, p<0.001).

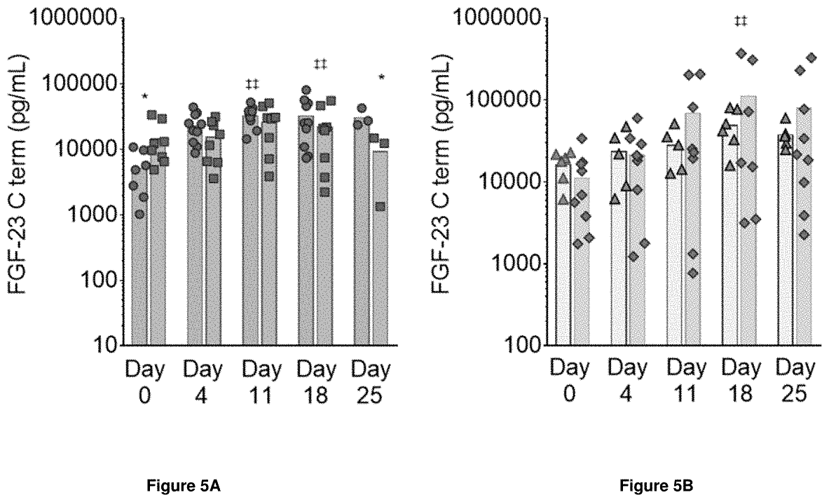

The results illustrated in FIG. 5 show that FGF23 levels were not significantly altered with fermagate treatment.

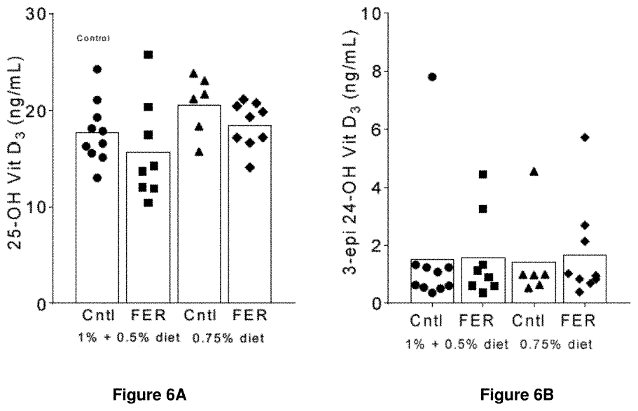

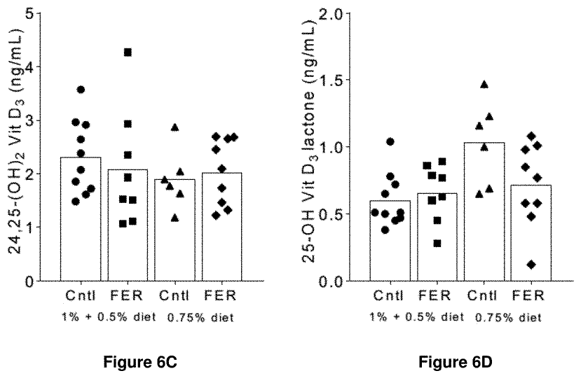

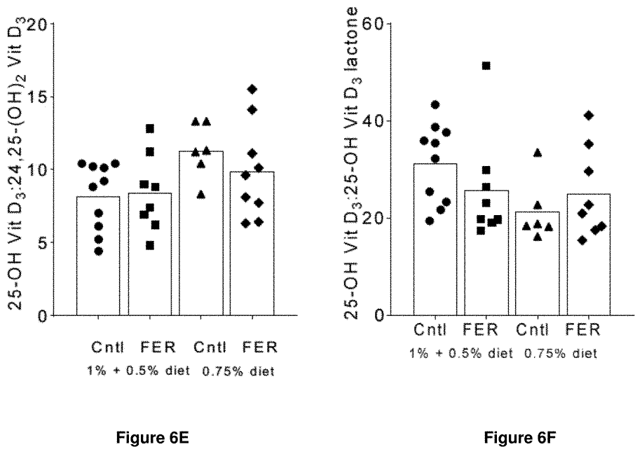

The results illustrated in FIG. 6 show that vitamin D profiles were similar between groups. Vitamin D levels were measured in sacrifice serum. Only controls 25-OH-D₃ lactone was different (p=0.02).

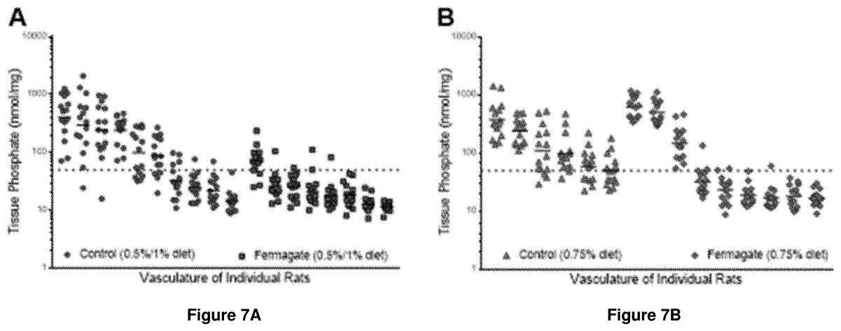

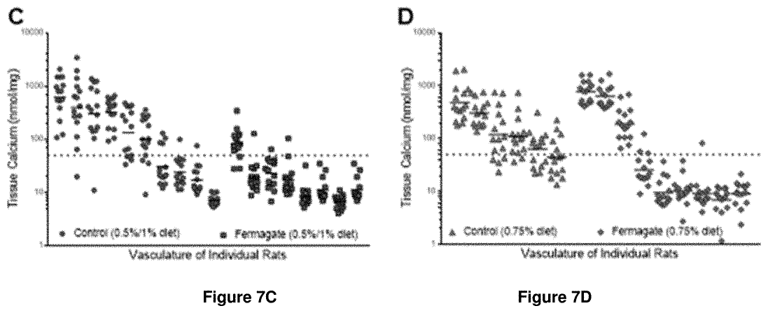

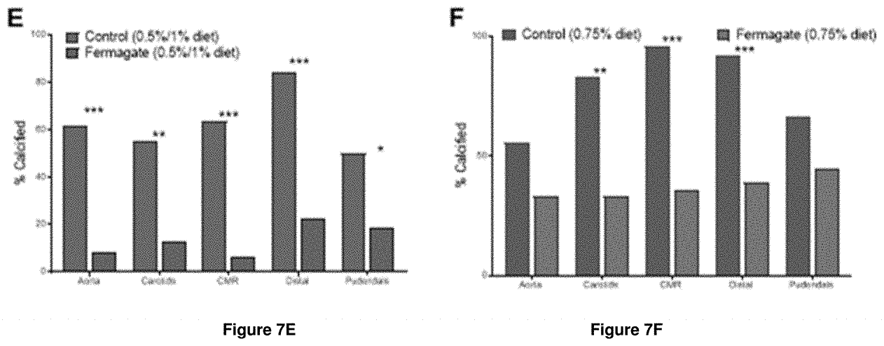

The results illustrated in FIG. 7 show that fermagate surprisingly prevented vascular calcification. The degree of vascular calcification was significantly reduced in arterial tissues with fermagate treatment (79%/65% in CON vs. 35% FER(0.75% P) and 13% FER(0.5-1% P), respectively, p<0.001). This inhibition was also evident on a per animal basis (100%/70% CON had VC vs. 33% FER(0.75% P) and 13% (FER 0.5-1% P), p<0.05, respectively).

In both studies, fermagate increased serum Mg (203% 0.75% P, p<0.0001; 163% 0.5-1% P, p<0.0001, % control, 2-way ANOVA) and had lower levels of serum PO4 (67% 0.75% P, p<0.001; 64% 0.5-1% P, p<0.001), and PTH (16% 0.75% P, p<0.001; 31% 0.5-1% P, p<0.001). The degree of VC was significantly reduced in arterial tissues with fermagate treatment (79%/65% in CON vs. 35% FER(0.75% P) and 13% FER(0.5-1% P), respectively, p<0.001). This inhibition was also evident on a per animal basis (100%/70% CON had VC vs. 33% FER(0.75% P) and 13% (FER 0.5-1% P), p<0.05, respectively). Fermagate treatment did not significantly alter Mg levels in the vasculature tissue, serum Ca, FGF23, or serum vitamin D metabolome.

These results demonstrate that fermagate effectively reduces the bioavailability of dietary PO₄, decreases serum PO₄ and PTH, increases serum Mg, and effectively limits the development and progression of CKD-induced vascular calcification.

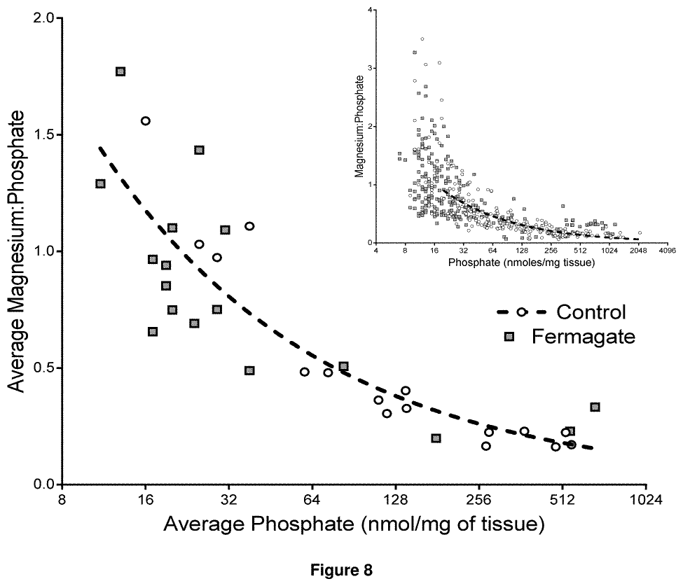

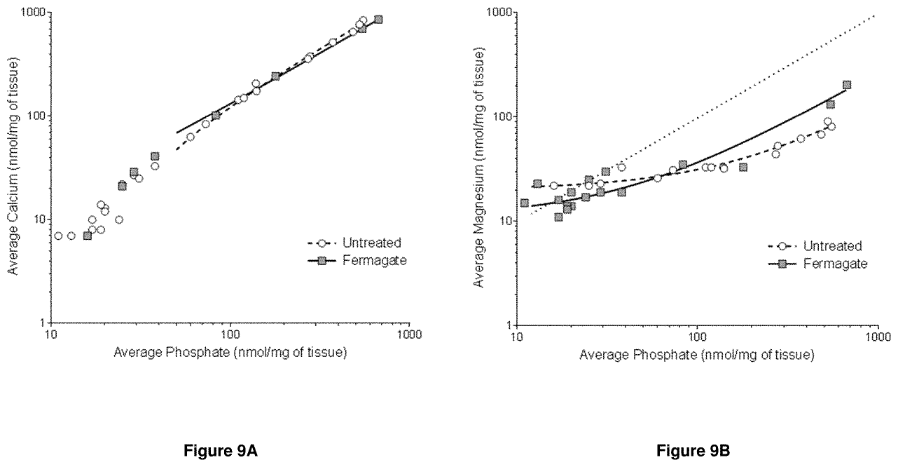

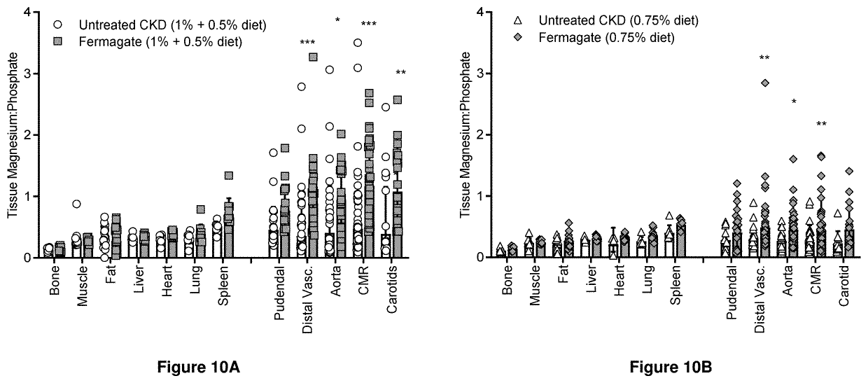

It was also observed that in the pathogenesis of crystal formation, there is a loss of magnesium incorporation per phosphate, that high serum magnesium is associated with the prevention of development of calcium phosphate crystal growth with most fermagate treatment, and that additional magnesium is not incorporated at similar rates in fermagate tissues with high amounts of calcium phosphate crystal. FIG. 8 shows the magnesium:phosphate ratio with phosphate concentration, with average per animal values shown in the main graph and individual tissues in the inset graph. FIG. 9 shows that magnesium is incorporated in the calcium deposit crystals at a lower ratio than Whitlockite, and similar to hydroxyapatite.

In both studies it was also observed that vascular tissues, but not other tissues or organs showed significantly magnesium accumulation relative to phosphate in animals treated with fermagate compared to untreated CKD animals. Only vascular tissues demonstrated a difference in magnesium to phosphate ratios. Bone did not show significant alternation of magnesium content between untreated CKD animals and those receiving fermagate. FIGS. 10A and 10B illustrate these results. In FIGS. 10A and 10B, each data point represents the ratio of magnesium to phosphate content of a single tissue from an individual rat. Tissues were ordered based on total average magnesium/phosphate for each tissue type across all treatments, with bone being the lowest and spleen highest of non-vascular tissues assessed. Tissues were as follows: bone (skull and tibia), muscle (quadriceps and abdominal), fat (subcutaneous and retroabdominal), liver, heart (left and right ventricle), lung, spleen. The arteries were also ordered, with pudendal being the lowest ratio and carotid the highest. Arteries were grouped: pudendal (two segments of the left pudendal artery), distal vasc. (left and right iliac, left and right femoral), aorta (thoracic, abdominal and arch), and carotids (left and right).

Comparisons were performed with two-way ANOVA and Sidak's multiple comparisons test using GraphPad PRISM 8.1.2. Treatment was significant for both studies (P<0.001) and the only tissues significantly different between fermagate and untreated control were arterial. For the 1%+0.5% diet study, the distal vascular (p<0.001), aorta (p<0.05), coronary CMR arteries (p<0.001), and carotid arteries (p<0.01) had significantly more magnesium to phosphate accumulation with fermagate treatment. For the 0.75% study, the distal vascular arteries (p<0.01), aorta (p<0.05), and coronary CMR arteries (p<0.01) had significantly more magnesium to phosphate accumulation with fermagate treatment.

The foregoing description is given for clearness of understanding only, and no unnecessary limitations should be understood therefrom, as modifications within the scope of the invention may be apparent to those having ordinary skill in the art.

Throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integers or steps.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

The practice of a method disclosed herein, and individual steps thereof, can be performed manually and/or with the aid of or automation provided by electronic equipment. Although processes have been described with reference to particular embodiments, a person of ordinary skill in the art will readily appreciate that other ways of performing the acts associated with the methods may be used. For example, the order of various of the steps may be changed without departing from the scope or spirit of the method, unless described otherwise. In addition, some of the individual steps can be combined, omitted, or further subdivided into additional steps.

1. A method of preventing and/or reducing vascular calcification, comprising:
administering to a subject in need thereof an effective amount of a mixed metal compound of formula (I):
M^{II}₁₋ₓ.M^{III}ₓ(OH)₂Aⁿ⁻_{y}.zH₂O, (I),
wherein M^{II} is at least one bivalent metal, M^{III} is at least one trivalent metal, Aⁿ⁻ is at least one n-valent anion, x=Σny, 0<x≤0.67, 0<y≤1, and 0≤z≤10.

2. A method of preventing and/or reducing vascular calcification, comprising:
administering to a subject in need thereof an effective amount of a mixed metal compound of formula (II):
M^{II}₁₋ₐM^{III}ₐO_{b}Aⁿ⁻_{c}.zH₂O (II),
wherein M^{II} is at least one bivalent metal; M^{III} is at least one trivalent metal; Aⁿ⁻ is at least one n-valent anion, 0<x≤0.67, 0<y≤1, and 0≤z≤10.

3. A method of preventing and/or reducing vascular calcification, comprising:
administering to a subject in need thereof an effective amount of a mixed metal compound of formula (VI):
M^{II}₁₋ₐM^{III}ₐO_{b}(Aⁿ⁻)_{c}.zH₂O (VI)
wherein M^{II} is at least one bivalent metal; M^{III} is at least one trivalent metal; and 1>a>0.4; 0<b≤2; 0<z≤5; Aⁿ⁻ is at least one n-valent anion; and 2+a−2b−cn=0.

4. A method of preventing and/or reducing vascular calcification, comprising:
administering to a subject in need thereof an effective amount of a mixed metal compound of formula (VII)
M^{II}₁₋ₐM^{III}ₐ(OH)_{d}](Aⁿ⁻)_{c}.zH₂O (VII)
wherein M^{II} is at least one bivalent metal; M^{III} is at least one trivalent metal; and 1>a>0.4; Aⁿ⁻ is at least one n-valent anion; 2+a−d−cn=0; Σcn<0.9a, 0≤d<2, and 0<z≤5.

5. The method of claim 1, wherein M^{II} comprises Mg.

6. The method of claim 1, wherein M^{II} is Mg.

7. The method of claim 1, wherein M^{III} comprises iron.

8. The method of claim 1, wherein Aⁿ⁻ comprises carbonate.

9. The method of claim 1, wherein M^{II} comprises magnesium, M^{III} comprises iron, and Aⁿ⁻ comprises carbonate.

10. The method of claim 1, wherein the mixed metal compound is substantially free of calcium.

11. The method of claim 1, wherein the subject in need thereof has hyperphosphatemia.

12. The method of claim 1, wherein the subject in need thereof has elevated FGF 23.

13. The method of claim 1, wherein the subject in need thereof has hyperphosphaturia.

14. The method of claim 1, wherein the subject in need thereof has recurrent urolithiasis.

15. The method of claim 1, wherein the subject in need thereof has idiopathic hypercalciuria.

16. The method of claim 1, wherein the subject in need thereof has hyperparathyroidism.

17. The method of claim 1, wherein the subject in need thereof has chronic kidney disease.

18. The method of claim 16, wherein the subject in need thereof has Chronic Kidney Disease Stage 3-5.

19. The method of claim 17, wherein the subject in need thereof has Chronic Kidney Disease Stage 3-4.

20. The method of claim 17, wherein the subject in need thereof has Chronic Kidney Disease Stage 5.

21. The method of claim 17, wherein the subject in need thereof has hyperparathyroidism secondary to Chromic Kidney Disease.

22. The method of claim 11, wherein the subject in need thereof does not have chronic kidney disease.

23. The method of claim 1, wherein the subject is human.

24. The method of claim 1, wherein upon administration the mixed metal compound releases the at least one bivalent metal and the at least one bivalent metal is preferentially absorbed by vascular tissue.

25. The method of claim 24, wherein the at least one bivalent metal is Mg.

26. The method of claim 25, comprising increasing the magnesium to phosphate accumulation in vascular tissue as compared to a control subject not receiving the mixed metal compound.

27. The method of claim 1, comprising administering at least about 200 mg of the mixed metal compound.

28. The method of claim 1, wherein the mixed metal compound is Mg4Fe2(OH)12CO3.nH2O, wherein n is 2 to 8.

29. The method of claim 1, wherein parathyroid hormone is reduced by at least 16%.

30. The method of claim 1, wherein a degree of vascular calcification is reduced to less than 40% vascular tissue calcified in the subject as compared to a control subject not receiving the mixed metal compound.

31. The method of claim 1, wherein vascular calcification is prevented in the subjects arterial tissue or heart tissue.

32.-62. (canceled)
